(19) United States
(12) Patent Application Publication
(10) Pub. No.: US 2007/0027460 A1
(43) Pub. Date: Feb. 1, 2007

(54) Implantable remodelable materials comprising magnetic material

(75) Inventors: Brian C. Case, Lake Villa, IL (US); Ram H. Paul JR., Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/493,065

(22) Filed: Jul. 26, 2006

Related U.S. Application Data
(60) Provisional application No. 60/703,288, filed on Jul. 27, 2005.

(51) Int. Cl. A61F 2/02

(52) U.S. Cl. 606/151; 600/431; 623/1.24

(57) ABSTRACT

Devices, materials and methods for using the same for modifying or monitoring a valve within a body are provided herein. Devices comprising a magnetic material and magnetically-activated implantable devices are described. Methods of modifying a valve in the body, for a valve within a body vessel are also provided.

### RELATED APPLICATIONS

This application claims the benefit of to U.S. Provisional Patent Application No. 60/703,288, entitled “Modification of a Valve in a Body Vessel to Improve Valve Function,” filed Jul. 27, 2005, which is incorporated herein by reference in its entirety.

### TECHNICAL FIELD

The present disclosure pertains to magnetic materials for implantation within a body vessel, as well as to methods of using the same to monitor or improve valve function within a body vessel.

### BACKGROUND

Devices and methods for modifying or monitoring valves within a body vessel are provided herein. In one aspect, methods and devices for modifying or monitoring the position or movement of leaflets of one or more venous valves are provided.

Many vessels in animals transport fluids from one body location to another. Frequently, fluid flows in a substantially unidirectional manner along the length of the vessel. For example, veins in the body transport blood to the heart and arteries carry blood away from the heart. Veins contain multiple venous valves to promote direction of blood flow back to the heart. Each venous valve is located inside the vein and typically contain at least two leaflets disposed annularly along the inside wall of the vein. The leaflets open to permit blood flow toward the heart and close, upon a change in blood pressure, such as during a transition from systole to diastole, to restrict the retrograde flow of blood. When blood flows towards the heart, an accompanying rise in fluid pressure against the valve leaflets forces the valve leaflets to move apart, thereby creating an open path for blood flow. When the fluid pressure against the leaflets subsides, the leaflets close to restrict or prevent retrograde blood flow, away from the heart. Properly functioning venous valve leaflets typically extend radially inward toward one another such that the distal ends of the leaflets contact each other when the venous valve is closed.

Valves within body vessels, such as venous valves, can fail to operate properly for a variety of reasons such as congenital valve deformation or degradation of valve tissue due to disease conditions. In the condition of venous valve insufficiency, venous valve leaflets do not function properly, failing to properly contact each other or failing to properly open or close in response to changes in fluid pressure. As a result of venous valve malfunction, increased levels of retrograde fluid flow in blood vessels can cause blood to pool in the lower extremities, which can lead to varicose veins or chronic venous insufficiency. If left untreated, venous valve insufficiency can cause venous stasis ulcers of the skin and subcutaneous tissue.

Valve leaflets in a body vessel become incompetent when the valve fails to function properly. With reference to FIG. 1A, a vein 10 comprises an incompetent venous valve 20 and a competent venous valve 25. Pulses of blood move through the vein 10 in the direction toward the heart 12, and a smaller amount of retrograde blood flow 16 moving in the direction away from the heart 14 between blood pulses. Each venous valve 20, 25 is a bicuspid valve, exemplified in the competent venous valve 25 by a first leaflet 26 and a second leaflet 27, and in the incompetent venous valve 20 by a first leaflet 21 and a second leaflet 22. When functioning properly, venous valves 20, 25 substantially block the retrograde flow 16.

In the incompetent venous valve 20, the leaflets 21, 22 fail to close properly to substantially block blood flow in a distal direction 14 away from the heart, permitting an undesirable amount of retrograde fluid flow 16 to pass through the body vessel 10. There are various anatomical causes for venous valve incompetence. For example, one or more leaflets can become improperly shaped or the leaflet tissue may become too stiff and fail to respond adequately to changes in fluid flow. Incompetent venous valve leaflets may fail to adequately contact each other at one or more locations. FIG. 1C shows a cross section of the incompetent venous valve 20 along the line A-A′ shown in FIG. 1A subjected to retrograde fluid flow 16 in a distal direction 14. The incompetent venous valve 20 fails to close, leaving an opening 24 between the first leaflet 21 and the second leaflet 22, both of which extend from to the interior wall 11 of the body vessel 10. The opening 24 allows an undesirable amount of retrograde fluid flow 16 to pass through the incompetent venous valve 20 in the distal direction 14.

FIG. 1B shows a cross section of the competent venous valve 25 along the line B-B′ shown in FIG. 1A blocking retrograde fluid flow 16 in a distal direction 14. In the competent venous valve 25, the first leaflet 26 and the second leaflet 27 move within the vein 10 lumen in response to fluid within the vein 10. Portions of the first leaflet 26 and the second leaflet 27 contact each other along a valve closure interface 29 to substantially block blood flow in the distal direction 14 away from the heart. Retrograde fluid flow 16 can collect in a first reservoir 30a defined by a portion the first leaflet 26 and the vein interior wall 11, exerting radially inward pressure on the first leaflet 26. A similar second reservoir 30b is formed by a portion of the second leaflet 27 and the vein interior wall 11. When blood flows in a proximal direction 12 toward the heart, the first leaflet 26 and the second leaflet 27 open toward the vein interior wall 11. The competent venous valve 25 closes along the valve closure interface 29, formed by contact between portions of the first leaflet 26 and the second leaflet 27, both of which extend from to the interior wall 11 of the body vessel 10.

In some circumstances, valves with impaired function can be replaced by implantation of a prosthetic valve. Implantable prosthetic valves can comprise magnetic material to facilitate desirable opening and closing dynamics of valve leaflets, for example as described in U.S. Pat. No. 4,417,360, filed Jul. 31, 1981, and U.S. Pat. No. 4,245,360, filed Jan. 24, 1979, both issued to Moasser. Prosthetic valves comprising magnetized leaflets are also described in U.S. Pat. No. 4,769,032, filed Mar. 5, 1986, to Steinberg. Recently, various implantable medical devices and minimally invasive methods for implantation of these devices have been developed to deliver these medical devices within the lumen of a body vessel. These devices are advantageously inserted intravascularly, for example from an implantation catheter. Such devices can comprise a one or more surfaces adapted for adhesion to a venous valve leaflet, a body vessel wall or both. For example, magnetic clips for heart valve repair are described by Published U.S. Patent Application No. US2004/0220593, filed Apr. 19, 2004 and published Nov. 4, 2004, by Greenhalgh. What is needed are effective non invasive devices and techniques to monitor valve function or to correct improper valve function, such as monitoring venous valve function and correcting incompetent venous valve leaflets by promoting closure of opposable valve leaflets.

### SUMMARY

Devices, materials and methods relating to modifying or monitoring a valve within a body are provided herein. Preferably, moveable portions of a valve inside a body vessel are modified or monitored by attaching the implantable devices, materials disclosed herein to moveable portions of the valve. Implantable devices can comprise magnetic materials or resilient materials that function to promote the beneficial opening or closing of valves. Valves can be maintained in a open or closed configuration in a releasable manner, permitting the valve to open or close in response to fluid flow contacting the valve in a body vessel, or in a non-releasable manner. Two or more implantable devices comprising magnetic materials can be positioned within a body vessel to promote the desirable closing or opening of the valve, for example by magnetically attracting or repelling moveable portions of the valve toward or away from each other. Implantation of medical devices by attachment to moveable portions of valves, such as valve leaflets, can also permit monitoring of the valve function by detection of the movement of the implanted magnetic materials within a body vessel. Any body valve can be modified or monitored using the devices and methods disclosed herein.

In one embodiment, intraluminally implantable laminar devices comprising a magnetic material attached to another material, including remodelable or synthetic polymer-based material, are provided. A laminar device can be formed from a suitable biocompatible synthetic polymer and a magnetic material, such as magnetic particles. A remodelable material can be selected to form the implantable laminar devices to allow for, and even promote, the ingrowth of cells into the device when placed in contact with living tissue. An extracellular matrix material is one preferred type of remodelable material, such as small intestine submucosa. A magnetic material of any suitable type or configuration is preferably fixed to the remodelable material in a manner that maintains the attachment of the magnetic material to the remodelable material when contacted with water or a body fluid. Iron oxides or magnetite are examples of suitable magnetic materials. Optionally, the magnetic material can be enclosed in a suitable coating material, including a synthetic bioabsorbable polymer such as polylactic acid or a non-bioabsorbable polymer such as a polyurethane, or combinations thereof. A magnetic material in any suitable structure can be employed. In one aspect, the magnetic material is a laminar patch of remodelable material impregnated with microparticles of magnetic particles. In another aspect, the magnetic material is a woven fabric of threads of a remodelable or synthetic polymer material with wires comprising a magnetic material. An implantable laminar device comprising magnetic material can be implanted to promote remodeling, to magnetically attract or repel portions of a valve within a body vessel, or to monitor the movement of a valve within in a body vessel.

In another embodiment, devices comprising a magnetically-activated valve modifying means are provided. Preferably, the devices are moveable between an inactive configuration and a valve-modifying configuration, and comprise a magnetically-actuated means for converting the device from the inactive configuration to the valve modifying configuration. In one aspect, the magnetically activated valve modifying means comprises a first strut joined to a second strut in operative communication with a magnetically-moveable releasing means for permitting the device to move from the inactive configuration to the valve modifying configuration. Preferably, the first strut is resiliently compressed to expand away from the second strut when released by the magnetically-moveable releasing means.

In another embodiment, methods of modifying a valve in the body are provided. Preferably, a valve in a body vessel is modified by desirably promoting the opening or closing of moveable portions of the valve, such as opposable leaflets of a venous valve or a heart valve. Medical devices can be implanted to exert force on moveable portions of a valve, such as opposable valve leaflets, to promote the relative motion of the moveable portions toward or away from each other. For example, two laminar magnetic devices can be separately attached to opposable valve leaflets such that the magnetic devices are attracted toward each other across a valve orifice (promoting closing of the valve), or are repelled from each other (promoting opening of the valve). The strength of attraction of pairs of implanted magnetic devices attached to opposable valve leaflets with respect to one another can be selected to provide a desired strength of closure of the valve. A weaker attraction between a pair of opposably positioned magnetic devices can permit the valve to open in response to fluid flow, while a strong attraction between the opposably positioned magnetic devices can divert fluid flow around the portion of the valve orifice where the magnetic devices are positioned.

Preferably, the intraluminally implantable device or material comprises a magnetic material, a resilient material or any combination thereof. In one aspect, a magnetic remodelable material or structure may be implanted in a body vessel, including any material disclosed in the first embodiment. In another aspect, a resilient material such as a superelastic NiTi alloy can be implanted in the body vessel. In yet another aspect, magnetic particles can be attached to portions of a body vessel or a valve within the body to modify a valve within the body. For example, magnetic microparticles adapted to attach to portions of the surface of the body vessel or valve can be implanted using a catheter.

Preferably, the resilient material is a self-expanding material capable of significant recoverable strain to assume a low profile for delivery to a desired location within a body lumen. After release of the compressed self-expanding resilient material, it is preferred that the frame be capable of radially expanding back to its original diameter or close to its original diameter. Accordingly, some embodiments provide frames made from material with a low yield stress (to make the frame deformable at manageable balloon pressures), high elastic modulus (for minimal recoil), and is work hardened through expansion for high strength. Particularly preferred materials for self-expanding implantable frames are shape memory alloys that exhibit superelastic behavior, i.e., are capable of significant distortion without plastic deformation. Frames manufactured of such materials may be significantly compressed without permanent plastic deformation, i.e., they are compressed such that the maximum strain level in the resilient material is below the recoverable strain limit of the material. Discussions relating to nickel titanium alloys and other alloys that exhibit behaviors suitable for frames can be found in, e.g., U.S. Pat. No. 5,597,378 (Jervis) and WO 95/31945 (Burmeister et al.). A preferred shape memory alloy is Ni—Ti, although any of the other known shape memory alloys may be used as well. Such other alloys include: Au—Cd, Cu—Zn, In—Ti, Cu—Zn—Al, Ti—Nb, Au—Cu—Zn, Cu—Zn—Sn, CuZn—Si, Cu—Al—Ni, Ag—Cd, Cu—Sn, Cu—Zn—Ga, Ni—Al, Fe—Pt, U—Nb, Ti—Pd—Ni, Fe—Mn—Si, and the like. These alloys may also be doped with small amounts of other elements for various property modifications as may be desired and as is known in the art. Nickel titanium alloys suitable for use in manufacturing implantable frames can be obtained from, e.g., Memory Corp., Brookfield, Conn. One suitable material possessing desirable characteristics for self-expansion is Nitinol, a Nickel-Titanium alloy that can recover elastic deformations of up to 10 percent. This unusually large elastic range is commonly known as superelasticity.

Materials and devices can be implanted within a body vessel at any suitable orientation or position. Preferably, a device can be implanted in contact with a portion of a valve within the body. In some aspects, an implantable device comprising a magnetic material and a remodelable material is transluminally implanted within a body vessel. The magnetic remodelable material is preferably delivered using a catheter based delivery system within a body vessel. In one aspect, an implant is positioned in contact with a portion of a valve in a body vessel, such as a valve leaflet of a venous valve or heart valve. In another aspect, two or more magnetic remodelable material devices can be implanted, simultaneously or sequentially, in contact with two or more portions of a valve or body vessel that are moveable with respect to one another. For example, a first magnetic remodelable material can be implanted in contact with a first valve leaflet; a second magnetic remodelable material can preferably be implanted in contact with a second valve leaflet that is opposable to the first leaflet, or in contact with a portion of the wall of the body vessel. In other aspects, a device comprising a resilient material such as a superelastic NiTi alloy is implanted in contact with a moveable portion of a valve. In one aspect, a device comprising a resilient alloy is implanted with a first surface of the device contacting a valve leaflet, the first surface joined to a second surface, with the second surface contacting a portion of the vessel wall. Preferably, the first surface of the device is adapted to hingeably move relative to the second surface.

In other embodiments, methods of monitoring the movement of a valve within a body vessel are provided. A method of monitoring the movement of a valve in a body vessel preferably comprises the steps of: implanting a magnetic material in moveable contact with a leaflet of the valve, and detecting the movement of the magnetic material.

While certain embodiments disclosed herein relate to the modification or modification of venous valve function within a body vessel, the invention is not limited to venous valve modification or monitoring. Non-limiting examples of suitable valves include any valves with leaflets, such as bicuspid calf valves and tricuspid valves such as heart valves. Embodiments are also provided that relate to monitoring or modifying the function of previously implanted prosthetic valves in any body vessel.

### BRIEF DESCRIPTION OF THE DRAWINGS

In the accompanying drawings:

FIG. 1A is a cut-away view of a segment of a body vessel comprising two bicuspid valves.

FIG. 1B is a cross sectional view of a first bicuspid valve in the body vessel shown in FIG. 11A.

FIG. 1C is a cross sectional view of a second bicuspid valve in the body vessel shown in FIG. 1A.

FIG. 2A is a side view of an implantable medical device comprising a magnetic material.

FIG. 2B is a top view of the implantable medical device of FIG. 2A.

FIG. 3A is a cut-away view of a magnetically-activated medical device in the inactive configuration within a body vessel at the site of a bicuspid valve.

FIG. 3B is a cut-away view of a magnetically activated medical device in the leaflet closing configuration within a body vessel at the site of a bicuspid valve, with an activating probe within the body vessel.

FIG. 4A is a cut-away view of a segment of a body vessel showing a pair of medical devices comprising a superelastic material positioned within a body vessel to press two leaflets of a bicuspid valve toward the closed position.

FIG. 4B is a cross sectional view of the pair of the medical devices in the body vessel shown in FIG. 8A.

FIG. 5A is a cut-away view of a segment of a body vessel showing a pair of medical devices in contact with two leaflets of a bicuspid valve.

FIG. 5B is a cross sectional view of the pair of medical devices connecting portions of two leaflets of the bicuspid valve in the body vessel shown in FIG. 5A.

FIG. 5C is a cross sectional view of the pair of medical devices releasably connecting portions of two leaflets of the bicuspid valve in the body vessel shown in FIG. 5A.

FIG. 6 is a cut-away view of a segment of a body vessel showing a plurality of medical devices occluding a body vessel at the site of two leaflets of a bicuspid valve.

FIG. 7 is a cut-away view of a segment of a body vessel showing two sets of medical devices releasably connecting two leaflets of a bicuspid valve.

FIG. 8 is a cut-away view of a segment of a body vessel showing a plurality of medical devices attached to the inner wall of a body vessel near the site of two leaflets of a bicuspid valve.

FIG. 9 is a cut-away view of a segment of a body vessel showing a pair of medical devices in contact with two leaflets of a bicuspid valve, and a catheter probe for monitoring the movement of the two leaflets.

FIG. 10A shows a first catheter delivery system during implantation of a pair of medical devices comprising a magnetic material to a bicuspid valve within a body vessel.

FIG. 10B shows the first catheter delivery system of FIG. 10A after implantation of a pair of medical devices comprising a magnetic material to a bicuspid valve within a body vessel.

FIG. 11 shows a second catheter delivery system during implantation of magnetic microparticles within a portion of a body vessel.

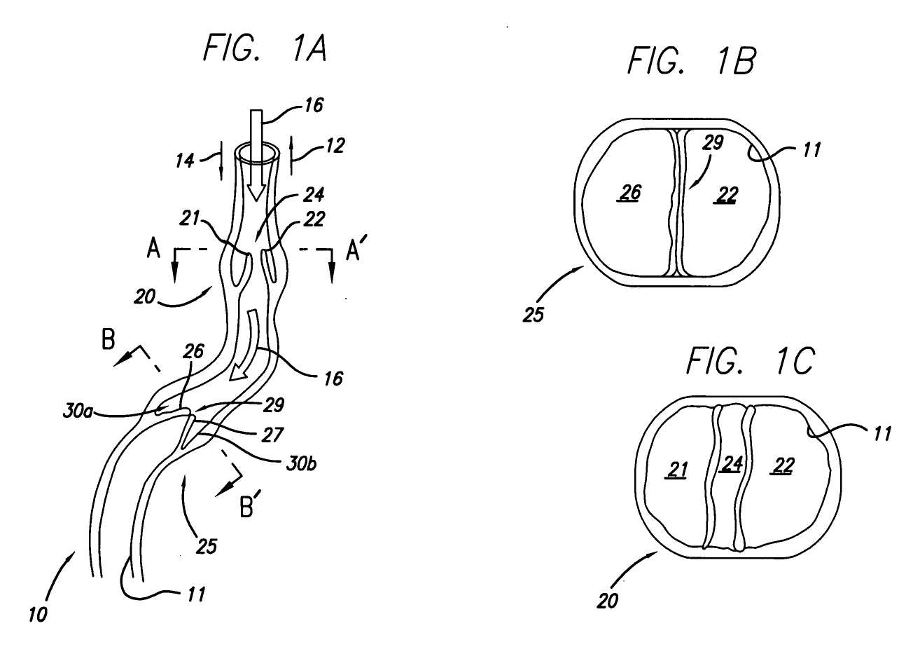

### DETAILED DESCRIPTION

Various devices for and methods of improving the function of incompetent valves are provided by the following illustrative embodiments. Preferably, the devices and methods disclosed improve the function of incompetent valves to more closely resemble the operation of a competent valve. In some embodiments, devices and methods related to modifying incompetent venous valves are provided, for example by magnetically attracting opposable incompetent venous valve leaflets toward the center of a vein lumen, to promote closure of a valve orifice or to maintaining portions of incompetent venous valve leaflets in contact with each other.

The following detailed description and appended drawings describe and illustrate various exemplary embodiments. Various medical devices for implantation in a body vessel, methods of making the medical devices, and methods of treatment that utilize the medical devices are provided herein.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

As used herein, “endolumenally” or “transluminal” mean placement by procedures wherein the prosthesis is advanced within and through the lumen of a body vessel from a remote location to a target site within the body vessel. In vascular procedures, a medical device will typically be introduced “endovascularly” using a catheter over a guidewire under fluoroscopic guidance. The catheters and guidewires may be introduced through conventional access sites to the vascular system, such as through the femoral artery, or brachial and subclavian arteries, for access to the coronary arteries.

As used herein, “laminar” medical devices refer to medical devices that have a thin substantially flat planar structure. A laminar device can have a thickness of between about 0.01 mm to about 5.00 mm, more preferably between about 0.025 mm to about 2.00 mm, most preferably between about 0.050 to about 0.50 mm.

As used herein, “bioabsorbable polymer” refers to a polymer or copolymer which is absorbed by the body.

A “biocompatible” material is a material that is compatible with living tissue or a living system by not being toxic or injurious and not causing immunological rejection.

“Non-bioabsorbable” material refers to a material, such as a polymer or copolymer, which remains in the body without substantial bioabsorption.

The recitation of a “first” direction is provided as an example. Any suitable orientation or direction may correspond to a “first” direction. The medical devices of the embodiments described herein may be oriented in any suitable absolute orientation with respect to a body vessel. For example, the first direction can be a radial direction in some embodiments.

The terms “proximal” and “distal” are used to connote a direction or position relative to a human body. In the vasculature, proximal and distal refer to the flow of blood to the heart, or away from the heart, respectively.

Implantable Magnetic Materials and Devices

In a first embodiment, a remodelable magnetic material is provided. Remodelable magnetic materials comprising a magnetic material attached to a remodelable material are provided. The magnetic remodelable materials can have any suitable shape and composition for an intended use. Preferably, the remodelable magnetic material is configured as an implantable medical device for magnetically attracting a leaflet toward another implanted magnetic surface. The medical device can be a very thin, highly flexible laminar patch that is sufficiently flexible to move with a portion of a valve leaflet surface within the body in response to fluid flow in the body vessel. Alternatively, an implantable magnetic device for maintaining portions of valve material in contact within a body vessel can be more rigid than the valve material.

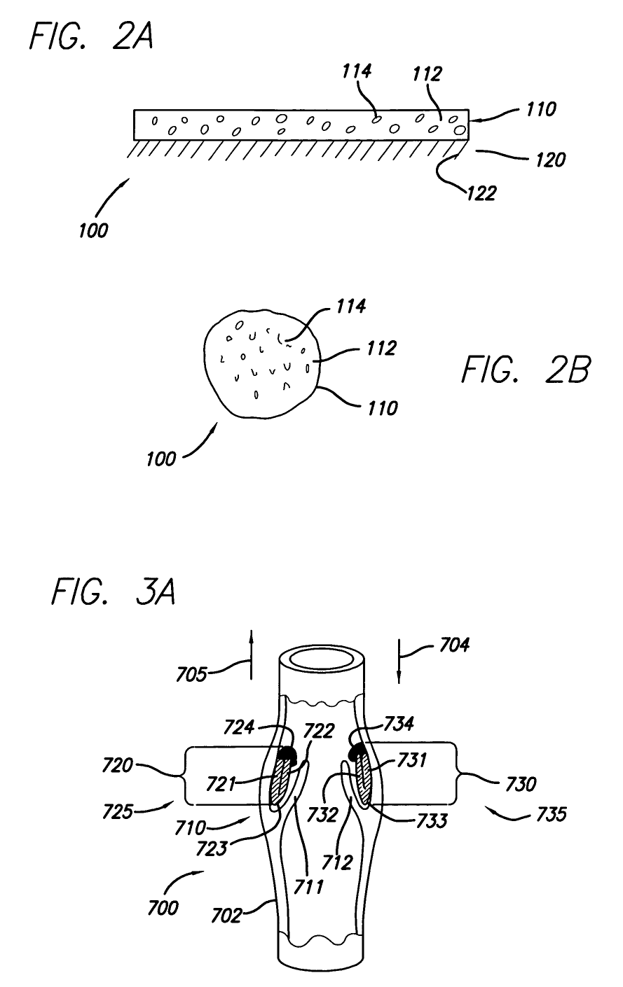

In one aspect, the device is a flexible laminar patch comprising a remodelable material, such as an extracellular matrix material, impregnated with magnetic material. FIG. 2A shows a cross section of a flexible laminar patch 100 having a first layer 110 and a second layer 120. The first layer 110 comprises an extracellular matrix material 112 derived from small intestine submucosa impregnated with a plurality of magnetic particles 114. The second layer 120 comprises a plurality of barbs 122. FIG. 2B shows a top view of the device 100 of FIG. 2A showing the first layer 110. Alternatively, the first layer 110 can be formed from different magnetic materials without a remodelable material. The first layer 110 can also be formed from woven strands of magnetic material. In another aspect, the first layer 110 is formed from a thin sheet of magnetic material. In yet another aspect, the laminar patch 100 is a single-layer construct comprising an extracellular matrix material 112 combined with a magnetic material, such as the magnetic particles 114.

The laminar patch 100 preferably has a thickness of between about 0.0001 inch and about 0.0030 inch, and more preferably about 0.0005 inch thick. The thickness can be measured by any conventional technique, including a conventional micrometer. Preferably, a venous valve leaflet has a variation in thickness of about 20%, more preferably about 10%, or less.

Remodelable Materials

The remodelable material is preferably selected to allow for the ingrowth of cells when placed in contact with living tissue. The terms “remodelable” or “bioremodelable” as used herein refer to the ability of a material to allow or induce host tissue growth, proliferation or regeneration following implantation of the tissue in vivo. Remodeling can occur in various microenvironments within a body, including without limitation soft tissue, a sphincter muscle region, body wall, tendon, ligament, bone and cardiovascular tissues. Upon implantation of a remodelable material, cellular infiltration and neovascularization are typically observed over a period of about 5 days to about 6 months or longer, as the remodelable material acts as a matrix for the ingrowth of adjacent tissue with site-specific structural and functional properties. The remodeling phenomenon which occurs in mammals following implantation of submucosal tissue includes rapid neovascularization and early mononuclear cell accumulation. Mesenchymal and epithelial cell proliferation and differentiation are typically observed by one week after in vivo implantation and extensive deposition of new extracellular matrix occurs almost immediately.

Remodelable materials can be intraluminally implanted within a body cavity, such as a blood vessel or organ, using percutaneous transcatheter techniques. The implanted remodelable material can be attached to a frame to form a valve or flow modifying device, or can be implanted without a frame. In either case, the remodelable material can be isolated and prepared by various techniques.

A remodelable material, can undergo biological processes such as angiogenesis when placed in communication with a living tissue, such that the remodelable material is biologically transformed into material that is substantially similar to said living tissue in cellular composition. Unless otherwise specified herein, a “remodelable material” can include a single layer material, or multiple layers of one or more materials that together undergo remodeling when placed in communication with living tissue. Preferably, a remodelable material undergoes a desired degree of remodeling upon contact for about 90 days or less with living tissue of the type present at an intended site of implantation, such as the interior of a body vessel.

One example of a remodeling process is the migration of cells into the remodelable material. Migration of cells into the remodelable material can occur in various ways, including physical contact with living tissue, or recruitment of cells from tissue at a remote location that are carried in a fluid flow to the remodelable material. In some embodiments, the remodelable material can provide an acellular scaffold or matrix that can be populated by cells. The migration of cells into the remodelable material can impart new structure and function to the remodelable material. In some embodiments, the remodelable material itself can be absorbed by biological processes. In some embodiments, fully remodeled material can be transformed into the living tissue it is in contact with through cellular migration from the tissue into the remodelable material, or provide the structural framework for tissue. Non-limiting examples of remodelable materials, their preparation and use are also discussed herein.

Any remodelable material, or combination of remodelable materials can be used as a remodelable material for practicing the present invention. For instance, naturally derived or synthetic collagen can provide retractable remodelable materials. Naturally derived or synthetic collagenous material, such as extracellular matrix material, are suitable remodelable materials. Examples of remodelable materials include, for instance, submucosa, renal capsule membrane, dura mater, pericardium, serosa, and peritoneum or basement membrane materials. Collagen can be extracted from various structural tissues as is known in the art and reformed into sheets or tubes, or other shapes. The remodelable material may also be made of Type III or Type IV collagens or combinations thereof. U.S. Pat. Nos. 4,950,483, 5,110,064 and 5,024,841 relate to such remodelable collagen materials and are incorporated herein by reference. Further examples of materials useful as remodelable materials include: compositions comprising collagen matrix material, compositions comprising epithelial basement membranes as described in U.S. Pat. No. 6,579,538 to Spievack, the enzymatically digested submucosal gel matrix composition of U.S. Pat. No. 6,444,229 to Voytik-Harbin et al., materials comprising the carboxy-terminated polyester ionomers described in U.S. Pat. No. 5,668,288 to Storey et al., collagen-based matrix structure described in U.S. Pat. No. 6,334,872 to Termin et al., and combinations thereof. In some embodiments, submucosal tissues for use as remodelable materials include intestinal submucosa, stomach submucosa, urinary bladder submucosa, and uterine submucosa. A specific example of a suitable remodelable material is intestinal submucosal tissue, and more particularly intestinal submucosa delaminated from both the tunica muscularis and at least the tunica mucosa of warm-blooded vertebrate intestine.

One preferred type of remodelable material is extracellular matrix material derived from submucosal tissue, called small intestine submucosa (SIS). Additional information as to submucosa materials useful as ECM materials herein can be found in U.S. Pat. Nos. 4,902,508; 5,554,389; 5,993,844; 6,206,931; 6,099,567; 6,358,284 and 6,375,989, as well as published U.S. Patent Applications US2004/0180042A1 and US2004/0137042A1, which are all incorporated herein by reference. For example, the mucosa can also be derived from vertebrate liver tissue as described in WIPO Publication, WO 98/25637, based on PCT application PCT/US97/22727; from gastric mucosa as described in WIPO Publication, WO 98/26291, based on PCT application PCT/US97/22729; from stomach mucosa as described in WIPO Publication, WO 98/25636, based on PCT application PCT/US97/23010; or from urinary bladder mucosa as described in U.S. Pat. No. 5,554,389; the disclosures of all are expressly incorporated herein.

The remodelable material can be isolated from biological tissue by a variety of methods. In general, a remodelable material such as an extracellular matrix (ECM) material can be obtained from a segment of intestine that is first subjected to abrasion using a longitudinal wiping motion to remove both the outer layers (particularly the tunica serosa and the tunica muscularis) and the inner layers (the luminal portions of the tunica mucosa). Typically the SIS is rinsed with saline and optionally stored in a hydrated or dehydrated state until use as described below. The resulting submucosa tissue typically has a thickness of about 100-200 micrometers, and may consist primarily (greater than 98%) of acellular, eosinophilic staining (H&E stain) ECM material.

Preferably, the source tissue for the remodelable material is disinfected prior to delamination by using the preparation disclosed in U.S. Pat. No. 6,206,931, filed Aug. 22, 1997 and issued Mar. 27, 2001 to Cook et al., and US Patent Application US2004/0180042A1 by Cook et al., filed Mar. 26, 2004, published Sep. 16, 2004 and incorporated herein by reference in its entirety. Most preferably, the tunica submucosa of porcine small intestine is processed in this manner to obtain the ECM material. This method is believed to substantially preserve the aseptic state of the tela submucosa layer, particularly if the delamination process occurs under sterile conditions. Specifically, disinfecting the tela submucosa source, followed by removal of a purified matrix including the tela submucosa, e.g. by delaminating the tela submucosa from the tunica muscularis and the tunica mucosa, minimizes the exposure of the tela submucosa to bacteria and other contaminants. In turn, this enables minimizing exposure of the isolated tela submucosa matrix to disinfectants or sterilants if desired, thus substantially preserving the inherent biochemistry of the tela submucosa and many of the tela submucosa's beneficial effects.

An alternative to the preferred method of ECM material isolation comprises rinsing the delaminated biological tissue in saline and soaking it in an antimicrobial agent, for example as disclosed in U.S. Pat. No. 4,956,178. While such techniques can optionally be practiced to isolate ECM material from submucosa, preferred processes avoid the use of antimicrobial agents and the like which may not only affect the biochemistry of the matrix but also can be unnecessarily introduced into the tissues of the patient. Other disclosures of methods for the isolation of ECM materials include the preparation of intestinal submucosa described in U.S. Pat. No. 4,902,508, the disclosure of which is incorporated herein by reference. Urinary bladder submucosa and its preparation is described in U.S. Pat. No. 5,554,389, the disclosure of which is incorporated herein by reference. Stomach submucosa has also been obtained and characterized using similar tissue processing techniques, for example as described in U.S. patent application Ser. No. 60/032,683 titled STOMACH SUBMUCOSA DERIVED TISSUE GRAFT, filed on Dec. 10, 1996, which is also incorporated herein by reference in its entirety.

Preferably, the remodelable material has an endotoxin level of less than 12 endotoxin units per gram, such as the small intestine submucosal material described in U.S. Pat. No. 6,358,284, filed Jun. 2, 1999 and incorporated herein by reference. An “endotoxin,” as used herein, refers to a particular pyrogen which is part of the cell wall of gram-negative bacteria. A “bioburden,” as used herein, refers to the number of living microorganisms, reported in colony-forming units (CFU), found on and/or in a given amount of material. Illustrative microorganisms include bacteria, fungi and their spores. Endotoxins are continually shed from the bacteria and contaminate materials The tubular purified submucosa graft constructs of the present invention can be sterilized using conventional sterilization techniques including glutaraldehyde tanning, formaldehyde tanning at acidic pH, propylene oxide or ethylene oxide treatment, gas plasma sterilization, gamma radiation, electron beam, and peracetic acid sterilization. Sterilization techniques which do not adversely affect the mechanical strength, structure, and biotropic properties of the purified submucosa is preferred. For instance, strong gamma radiation may cause loss of strength of the sheets of purified submucosa. Preferred sterilization techniques include exposing the graft to peracetic acid, 1-4 Mrads gamma irradiation (more preferably 1-2.5 Mrads of gamma irradiation), ethylene oxide treatment or gas plasma sterilization; peracetic acid sterilization is the most preferred sterilization method. Typically, the purified submucosa is subjected two or more sterilization processes. After the purified submucosa is sterilized, for example by chemical treatment, the matrix structure may be wrapped in a plastic or foil wrap and sterilized again using electron beam or gamma irradiation sterilization techniques.

Magnetic Materials

Various magnetic materials may be implanted within a body vessel. Magnetic materials may be combined a remodelable material to form a magnetic remodelable material adapted for implantation within a body, for example to modify a valve within a body vessel. Alternatively, magnetic materials can be combined with a biocompatible synthetic polymer, such as a polyurethane, or a biodegradable polymer. Magnetic materials can also be combined with biomaterials, such as collagen. Magnetic materials may also be implanted as magnetic particles adapted to attach to portions of a valve within a body vessel.

A magnetic material may be temporary magnetic materials or permanent magnetic materials. Some examples of suitable magnetic materials include iron oxide, magnetite, or samarium cobalt, or ‘ferrite,’ which is a substance consisting of mixed oxides of iron and one or more other metals. One specific example of a suitable magnetic ferrite material is nanocrystalline cobalt ferrite; however other ferrite materials may be used. Other magnetic materials include but are not limited to: ceramic and flexible magnetic materials made from strontium ferrous oxide which may be combined with a polymeric substance such as plastic, or rubber; NdFeB (this magnetic material may also include Dysprosium); SmCo (Samarium, Cobalt); and combinations of aluminum, nickel, cobalt, copper, iron, titanium as well as other materials.

The magnetic material is preferably provided as a powder or particulate. The magnetic material can be coated on microparticles. Optionally, the magnetic material can be enclosed in or mixed with a suitable coating material, such as a bioabsorbable polymer, a non bioabsorbable polymer, or a biological material such as an extracellular matrix material. For example, a magnetic material can be a 1-2 μm polystyrene particle coated with a mixture of magnetic iron oxide (magnetite) and polystyrene, such as the paramagnetic particles sold under the tradename SPHERO™ Magnetic Particles (Spherotech, Inc., Libertyville, Ill.); the polystyrene polymer combined with the magnetite can optionally be cross linked to increase the surface area and magnetite content. Another suitable source of a magnetic material are magnetic ferrofluids comprising nanoparticles (ca. 1-100 nm) of iron oxides in a stable colloidal suspension in water at about 1.7-5.0 v %, such as the ferrofluid sold under the tradename Pure Precision™ available from FerroTec containing a mixture of 10 nm particles of Fe3O4 and γ-Fe2O3 iron oxides. Another suitable magnetic material is a thin film or particle comprising nanoparticles of ferrite coated styrene and methyl methacrylate polymer films, the preparation of which is described in I. Neamtu, et al., “Polymer-Coated Ferrite Nanocomposites Synthesized by Plasma Polymerization,” Rom. Journ. Phys. v. 50, nos. 9-10, pp. 1081-1087 (2005), incorporated herein by reference.

The strength of magnetic field can be chosen to provide desirable valve modification properties. For example, the magnetic field strength should be chosen to provide the beneficial effects desired. The magnetic materials chosen, the density of the magnetic materials, the orientation of the magnetic materials and other parameters can be chosen based on a variety of considerations including the location and function of the valve to be modified. Other factors relating to the valve such as fluid flow rates, fluid pressure, valve strength, and valve location within the body may also inform the selection of an appropriate magnetic field strength. For devices comprising magnetic materials for releasably closing portions of a body valve, a lower magnetic field strength may be selected than for devices comprising magnetic materials for connecting portions of a valve. In general, however, the magnetic materials or magnetic properties of an implantable device comprising a magnetic material preferably emit a magnetic field of between about 20 to 10,000 gauss and preferably between 400 and 2000 gauss. Desirably, the magnetic material has a field strength sufficient for an intended use, such as to attract or repel another sample of the magnetic material positioned within a body vessel.

Remodelable Magnetic Materials

A remodelable magnetic material is preferably formed by fixedly attaching a magnetic material to or within a remodelable material. The combination of the remodelable material with the magnetic material may enhance the remodeling process upon contacting the remodelable magnetic material with living tissue. Without being bound to theory, research has indicated that biophysical input such as exposure to electromagnetic fields may regulate the expression of genes in connective tissue cells for structural extracellular matrix (ECM) proteins, and may lead to stimulation of growth factors. R K Aaron et al., “Stimulation of growth factor synthesis by electric and electromagnetic fields,” Clin. Orthop., 419, 30-37 (February 2004), which is incorporated herein by reference in its entirety, reviews some research reports relating to the potentially beneficial effects of combining a magnetic material with a remodelable material. The composite materials comprising the ECM and magnetic materials is preferably configured with a thickness of between about 0.0001 inch and about 0.0050 inch, including thickness of 0.0040, 0.0030, 0.0020, 0.0010, 0.0008, 0.0006, 0.0005, 0.0004, 0.0003, and 0.0002-inch, and more preferably about 0.0030 to about 0.0005 inch thick.

A magnetic material of any suitable type or configuration is preferably attached to the remodelable material in a manner that maintains the attachment of the magnetic material to the remodelable material while in contact with a body fluid. A remodelable material can be attached to a magnetic material by any suitable method to form a magnetic remodelable material that retains a desirable level of magnetism and attachment to the remodelable material when the magnetic remodelable material is exposed to a suitable biological tissue environment. Recitation of the “attachment” of a magnetic material to a remodelable material herein refers broadly to any method of joining or configuration of the two materials, including attachment or coating of one material to the surface of another material, impregnation of one material into another, or a mixture of the two materials together in one or more layers. A suitable biological tissue environment can include any conditions encountered at a point of desirable implantation in the body, such as exposure to blood or tissue fluids, fluid flow conditions, temperatures or biologically active molecules typically found within a site of implantation.

According to a first preferred method for combining a magnetic material and a remodelable material, the magnetic material is intimately mixed with a fluidized remodelable material, which is then dried into a composite sheet to form remodelable magnetic material having a desired thickness. The fluidized remodelable compositions are prepared as solutions or suspensions of an extracellular matrix material (ECM) by comminuting and/or digesting the ECM with a protease, such as trypsin or pepsin, for a period of time sufficient to solubilize said tissue and form a substantially homogeneous solution. The ECM starting material can be comminuted by any suitable method (e.g., tearing, cutting, grinding, shearing and the like). Grinding the ECM in a frozen or freeze-dried state is preferred, although a suspension of pieces of the ECM can also be comminuted in a high speed (high shear) blender with dewatering, if necessary, by centrifuging and decanting excess water. The comminuted ECM can be dried to form an ECM powder. Thereafter, the ECM can be hydrated, by combining with water or buffered saline and optionally other pharmaceutically acceptable excipients to form a fluidized ECM composition. Optionally, the fluidized material may be subjected to proteolytic digestion to form a substantially homogeneous solution. In one embodiment, the ECM powder is digested with 1 mg/ml of pepsin (Sigma Chemical Co., St. Louis, Mo.) in 0.1 M acetic acid, adjusted to pH 2.5 with HCl, over a 48 hour period at room temperature. The reaction medium is neutralized with sodium hydroxide to inactivate the peptic activity. The solubilized ECM may then be concentrated by salt precipitation of the solution and separated for further purification and/or freeze drying to form a protease solubilized intestinal submucosa in powder form. The viscosity of fluidized ECM compositions can be manipulated by controlling the concentration of the ECM component and the degree of hydration. The viscosity can be adjusted to a range of about 2 to about 300,000 cps at 25.degree. C. Higher viscosity formulations, for example, gels, can be prepared from the SIS digest solutions by adjusting the pH of such solutions to about 6.0 to about 7.0. Additional details pertaining to the preparation of a fluidized ECM remodelable material are found in U.S. Pat. No. 5,275,826, filed Nov. 13, 1993 (Badylak et al.), incorporated herein by reference. One or more magnetic materials, such as powders, microparticles, nanoparticles, or magnetic beads or colloidal suspensions thereof, are preferably mixed with the fluidized ECM material described above. The mixture can be dried into a sheet having a desired thickness to form a laminar patch 100 shown in FIG. 2A.

Alternatively, the magnetic material can be pressed into one or more sheets of the remodelable material. For example, magnetic particles can be placed between two parallel sheets of small intestine submucosa, which are then pressed together and dried in any manner effective to join the two sheets to form a magnetic composite material. For example, the two sheets of small intestine submucosa can be tensionably compressed between two heated nip rollers to seal the magnetic material between the sheets.

In one preferred embodiment, the remodelable magnetic material may comprise a polyurethane, in combination with the remodelable material, such as small intestine submucosa (SIS), and the magnetic material. One example of a preferred biocompatible polyurethane is sold under the tradename THORALON (THORATEC, Pleasanton, Calif.). Descriptions of suitable biocompatible polyureaurethanes are described in U.S. Pat. Application Publication No. 2002/0065552 A1 and U.S. Pat. No. 4,675,361, both of which are incorporated herein by reference. Briefly, these publications describe a polyurethane base polymer (referred to as BPS-215) blended with a siloxane containing surface modifying additive (referred to as SMA-300). Base polymers containing urea linkages can also be used. The concentration of the surface modifying additive may be in the range of 0.5% to 5% by weight of the base polymer. The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference. The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED). The composition can contain up to about 40 wt % polymer, with different levels of polymer within the range can be used to adjust the viscosity for a given process. Compositions comprising higher levels of polymer (ca. 5% to 25% polymer) generally have higher viscosity and are suitable for application by dipping. The composition can also contain less than 5 wt % polymer for forming a low viscosity composition suitable for application by spraying.

A polyurethane can also comprise a variety of other biocompatible polyurethanes/polycarbamates and urea linkages (hereinafter “—C(O)N or CON type polymers”). These include CON type polymers that preferably include a soft segment and a hard segment. The segments can be combined as copolymers or as blends. For example, CON type polymers with soft segments such as PTMO, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

Optionally, a remodelable magnetic material can comprise a polyurethane cross-linked to an ECM such as small intestine submucosa. Cross linking of these two materials can be accomplished by reacting the ester functionality of SIS with a crosslinking agent containing an oxygen or nitrogen to form an ester or amide bond, respectively. Polyurethane ureas can be cross-linked by reaction of the urea functionality with an oxygen or nitrogen functionality to form a urea bond or urethane bond. Polyamines, polyalcohols or amino alcohols are suitable cross-linking agent to cross-link polyurethane ureas and SIS. Alternatively, an epoxy amine or epoxy alcohol could be used to cross-link a polyurethane and SIS. In this case the amine or alcohol functionality of the cross-linking agent would form an ester or amide bond with the SIS material, and the epoxy functionality of the crosslinking agent would alkylate the urea functionality.

Preferably, the composite material comprising the ECM and magnetic material can be cross-linked to strengthen the material. Cross-linking can be performed, for example, to mechanically stabilize the remodelable magnetic material. Cross-linked material generally refers to material that is completely cross-linked in the sense that further contact with a cross-linking agent does not further change measurable mechanical properties of the material. However, total (100%) cross-linking is not always needed to achieve many desired mechanical properties. Cross-linking of the material preferably involves a chemical cross-linking agent with a plurality of functional groups that bond to the material 30 to form a chemically cross-linked material 30. The chemical cross-linking is preferably performed until a cross-linking agent has permeated the material of the cross-linking region and reacted with the accessible binding sites of the material.

Cross-link bonds can be formed in any suitable manner that provides attachment of a material to an implantable frame, including formation of cross-link chemical bonds between two surfaces of the material and/or formation of a cross-link bond between the frame and a portion of material. For example, cross-linking can be introduced by chemical treatment of the frame and/or material, such as glycolylation. The material can be subjected to a form of energy to introduce cross-linking. For example, energy treatment suitable for use in the invention includes exposing the material to ultraviolet light, heat, or both. In general, the material for use in the medical device and material for leaflet formation can be processed prior to cross-linking the material. For example, the material can undergo cutting and trimming, sterilizing, and associating the material with one or more desirable compositions, such as anticalcification agents and growth factors, and the like. After any preliminary processing and or storage is completed, the material can be cross-linked. Following cross-linking of the material, the material can be further processed, which can involve additional chemical and or mechanical manipulation of the material as well as processing the material into the desired medical device. Other cross-linking agents can be used to form cross-linking regions, such as epoxides, epoxyamines, diimides and other difunctional polyfunctional aldehydes. In particular, aldehyde functional groups are highly reactive with amine groups in proteins, such as collagen. Epoxyamines are molecules that generally include both an amine moiety (e.g. a primary, secondary, tertiary, or quaternary amine) and an epoxide moiety. The epoxyamine compound can be a monoepoxyamine compound and or a polyepoxyamine compound. In some embodiments, the epoxyamine compound is a polyepoxyamine compound having at least two epoxide moieties and possibly three or more epoxide moieties. In some embodiments, the polyepoxyamine compound is triglycidylamine (TGA). The use of cross-linking agents forms corresponding adducts, such as glutaraldehyde adducts and epoxyamine adducts, of the cross-linking agent with the material that have an identifiable chemical structures.

Alternatively, materials may be cross-linked using radical reactions. A radical is generated in the material to be cross-linked using a free radical generator, such as an organic peroxide of which many are known and commercially available, such as dicumyl peroxide, benzoyl peroxide, and the like. In this case the crosslinking agent is a multifunctional monomer capable of crosslinking the particular polymer when initiated by the free radical generator or irradiation. Typically, the crosslinking agent contains at least two ethylenic double bonds, which may be present, for example, in allyl, methallyl, propargyl or vinyl groups.

According to a second preferred method for combining the magnetic material and the remodelable material, the remodelable magnetic material is formed as a Langmuir-Blodgett molecular thin film according to the Langmuir-Blodgett Technique. A Langmuir-Blodget (“L-B”) molecular thin film is a composite material comprising one or more molecular layers of a material deposited on a solid substrate. The deposited material or the solid substrate can be a magnetic material and/or a remodelable material. Preferably, the L-B film comprises a thin film of a magnetic material deposited on a remodelable material solid substrate. As recognized in the art, L-B films can be produced by a three-step process: (1) a Langmuir monolayer film of the deposited material is formed on the surface of a liquid, for example by spreading a hydrophobic liquid comprising the magnetic material on a hydrophilic liquid (or vice versa); (2) slowly compressing the Langmuir monolayer of the hydrophobic liquid to increase the density of the molecular packing at the gas-liquid interface; and (3) dipping the solid substrate into the monolayer in a manner permitting the monolayer to attach to the substrate, thereby forming a first layer of the deposited material on the surface of the substrate, and withdrawing the solid substrate in a manner permitting a second layer of the molecules in the Langmuir monolayer to adhere to the first layer of the deposited material. By repeating these steps, multi-layer films of magnetic materials can be deposited on a remodelable solid substrate surface. For example, L-B films comprising magnetic materials, such as metal oxalate complexes (iron-chromium) and mixed valent manganese clusters based on a Mn₁₂O₁₂ core have been deposited on other substrates. Additional description of these and other magnetic L-B films, and the preparation thereof, are provided in Christoph Mingotaud, et al., “Magnetic Langmuir Blodgett Films,” in Chapter 14 of Magnetism: Molecules to Materials II: Molecule-Based Materials,” Wiley-VCH© 2002, pp. 457-484, which is incorporated herein by reference in its entirety.

According to a third method for preparing magnetic remodelable materials, the magnetic material is chemically joined to proteins in the remodelable material. For example, a magnetic material can be obtained that is adapted for molecular coupling to proteins, such as those found in a remodelable material. For example, the amine-terminated magnetic beads sold under the trade name BcMag (BioClone Inc., San Diego, Calif.) are supplied as an aqueous suspension of magnetic iron oxide particles having primary amino groups on the surface (1-5 μm beads, 50 mg/ml in 1 mM EDTA, pH 7.0). The BcMag bead can be attached to a protein in a fluidized ECM by preparing a solution comprising 30-100 mg of the fluidized ECM and 10 mL of a suitable coupling buffer and mixed, per manufacturer's directions. Any suitable magnetic material can be combined with a suitable molecular moiety, such as primary amino groups, to bind to protein in a fluidized ECM material.

Methods for producing ferromagnetic microdisks can also be adapted to produce a ferromagnetic thin film on a thin sheet of ECM material to provide a remodelable magnetic material. One example of a method for producing ferromagnetic microdisks includes: V. Novosad et al., “Ferromagnetic Microdisks: Novel Magnetic Particles for Biomedical Applications,” Nanotech 2005 vol. 1, Technical Proceedings of the 2005 NSTI Nanotechnology Conference and Trade Show, Volume 1, Chapter 6: BioNano Materials.

Another method for fixedly attaching a magnetic material within a remodelable material is by impregnating magnetic material within the remodelable material. Impregnation of magnetic material within a remodelable material may be accomplished by ion implantation of magnetic material within the remodelable material. The procedure set forth generally in Picraux and Pearcy, “Ion Implanation of Surfaces,” Scientific American, (March 1985) pp. 102-112, and U.S. Pat. No. 4,769,032, both of which are incorporated by reference herein, provides one suitable way to fixedly attach a magnetic material to a remodelable material by implantation. Accordingly, ion implantation of a magnetic material is performed by electrostatically accelerating a beam of magnetizing ions into the surface of a remodelable material. In this way, a controllable quantity of almost any element can be mingled with a remodelable material. For example, a remodelable material made either of porcine or non-porcine tissue can be implanted with ions that are either magnetizing or conductive, to form a thin layer of substantially any desired configuration at any predetermined depth. More specifically, ions of the material to be implanted originate at one end of an accelerator in a chamber in which electrons boil from a heated filament. The ions are accelerated by electric fields. Another electric field draws the ions from the chamber. The beam of ions is focused and accelerated to high energies, typically between 10 and 500 kiloelectron volts. Just before the ions strike the target, varying electric fields created by charged plates deflect the beam to eliminate neutral particles and to sweep the beam across the target for a uniform surface treatment. It is possible to anticipate, and therefore to control, not only the depth and distribution of the implanted atoms, but also the change in composition (i.e. magnetic and conductive composition) which they produce in the host material. In this manner, magnetizing and/or conductive ions can be ion implanted in any desired configuration of lines, defining circles or areas on remodelable material, whether porcine or non-porcine, to create a magnetic remodelable material.

Magnetically-Activated Implantable Devices

In a second embodiment, magnetically-activated medical devices are provided. The magnetically-activated medical devices are preferably transluminally implanted within a body vessel in an inactive configuration, and later magnetically activated to close a valve within a body vessel. In one aspect, a magnetically activated valve closure means is provided. When activated, the valve closure means can be configured to exert force against a moveable portion of a valve in a body vessel, thereby moving a portion of the valve in a desirable manner.

Preferably, a magnetically-activated valve closure device is moveable between an inactive configuration and a valve-closing configuration, and comprises a magnetically-actuated, or magnetically-moveable, releasing means for converting the device from the inactive configuration to the valve-closing configuration. The valve closure means can include a first surface moveable with respect to a second surface. The first surface is preferably part of a strut that is resiliently compressed to exert pressure away from a second strut comprising the second surface, and the magnetically-moveable releasing means maintains the first strut at a first distance from the second strut in the inactive configuration. Also preferably, the second strut moves relative to the first strut to a second distance that is greater that the first distance in the valve-closing configuration.

A medical device in the inactive configuration can be implanted in a body vessel without a substantial change in the vessel function. The medical device in the inactive configuration can be activated after implantation, by action of a suitable magnetic field imposed on the magnetically-moveable releasing means, thereby converting the medical device to a second configuration that performs a desirable function relative to a valve within the body vessel. The magnetically-moveable releasing means can be any structure or combination of structures responsive to a magnetic field to allow the device to move from the inactive configuration to a second configuration, which can be a valve-closing configuration. Optionally, a magnetically-moveable releasing means can also retain the medical device in an inactive configuration. A medical device in the valve-closing configuration can maintain portions of a leaflet in fixed contact with each other, or releasably close portions of the leaflet together. The material, configuration and orientation of a medical device can be selected to provide an optimal level of resiliency to allow for the intended valve function when the device is in the valve-closing configuration.

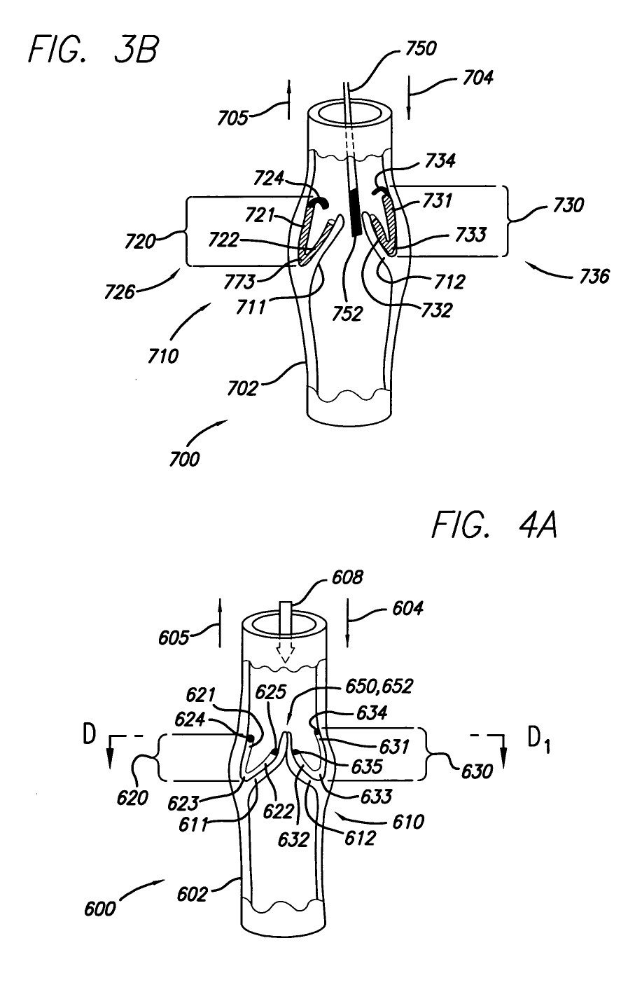

FIG. 3A shows a segment of a vein 700 comprising a venous valve 710. A pair of substantially identical magnetically-activated medical devices 720, 730 are deployed within the vein 700. Each medical device 720, 730 is a bent tube of superelastic material retained in a compressed, inactive configuration 725, 735 at a more acute angle within the vein 700 than the configuration each tube would assume without external restraint. The medical device 720 comprises a member 721 anchored to a portion of the vein wall 702 and resiliently joined by a bend 723 to a member 722 that does not continuously contact the first leaflet 711. In the inactive configuration 725 a magnetically-activated clasp 724 retains the member 721 in contact with the member 722. Similarly, in the inactive configuration 735, the medical device 730 comprises a member 731 anchored to a portion of the vein wall 702 and resiliently joined by a bend 733 to a member 732 that does not continuously contact the leaflet 712. In the inactive configuration 735 a magnetically-activated clasp 734 retains the first member 731 in contact with the second member 732. The magnetically-activated clasp can be made from any suitable material responsive to a magnetic field, such as a metal, conductor, or another magnetic material.

The amount of mechanical resilience of the medical devices 720, 730 can be calibrated to the intended function within the vein 700. Preferably, the medical devices 720, 730 are anchored to the wall of the vein 702 by any suitable means, including barb structures, adhesives or sealing using RF energy emitted from a catheter probe within the vein 700.

As shown in FIG. 3B, the medical devices 720, 730 have been converted to the valve closing configuration 726, 736 by exposure to a suitable magnetic field from the magnetic portion 752 of a catheter probe 750 temporarily positioned within the vein 700. Once in the valve-closing configuration 726, 736, the medical devices 720, 730 can be sufficiently resilient to compress the leaflets 711, 712 toward a closed position but allow the valve 710 to open in response to fluid flow in a first direction 705, while promoting closure of the valve 710 when fluid flows in the opposite direction 704. Alternatively, the medical devices 720, 730 can maintain the central portion of the leaflets 711, 712 together. While this embodiment describes medical devices with a tubular configuration, any suitable shape can be deployed, including disks, cylinders, spheres, laminar sheets, and others. Optionally, portions 722 and 732 of the medical devices 720 and 730, respectively, can include a means of attachment to a moveable portion of the valve, such as a valve leaflet, which can include barb structures, adhesives or sealing by application of RF energy emitted from a catheter probe.

In one aspect, a device for implantation in a body vessel comprises a magnetically activated valve modifying means moveable between an inactive configuration and a valve modifying configuration and having a magnetically-actuated means for converting the inactive configuration to the valve closing configuration. In one particularly preferred aspect, the device for implantation in a body vessel comprises a magnetically activated valve modifying means that is a device comprising a first strut having a proximal end and a distal end joined to a second strut having a proximal and a distal end. Preferably, the proximal end of the first strut is joined to the proximal end of the second strut. Also preferably, the distal end of the first strut has a first magnetic surface that is magnetically repelled to move away from a second magnetic surface on the distal end of the second strut. The magnetically-actuated means is preferably moveable between a retaining position and a release position in response to a magnetic field. The magnetically-actuated means in the retaining position preferably retains the distal end of the first strut at a first distance from the distal end of the second strut when the valve modifying means is in the inactive configuration. The distal end of the first strut preferably moves relative to the distal end of the second strut when the valve modifying means is moved from the retaining position to the release position.

Resilient Materials

In a third embodiment, medical devices comprising a resilient material such as a superelastic NiTi alloy, are provided with and without magnetic material. Medical devices comprising resilient materials can optionally comprise a magnetic material, and are preferably configured to modify valve function by exerting force on a portion of a valve or body vessel to promote desirable valve closure or valve opening. Medical devices comprising resilient materials are preferably implanted in the body vessel, for example to releasably close portions of two opposable valve leaflets or to connect two or more valve leaflets or portions thereof. Releasable closure of opposable valve leaflets provides a closed valve orifice when fluid flows in a retrograde direction, but permits the valve orifice to open in response to fluid flow in a desired direction. The desired direction for fluid flowing through a venous valve is toward the heart.

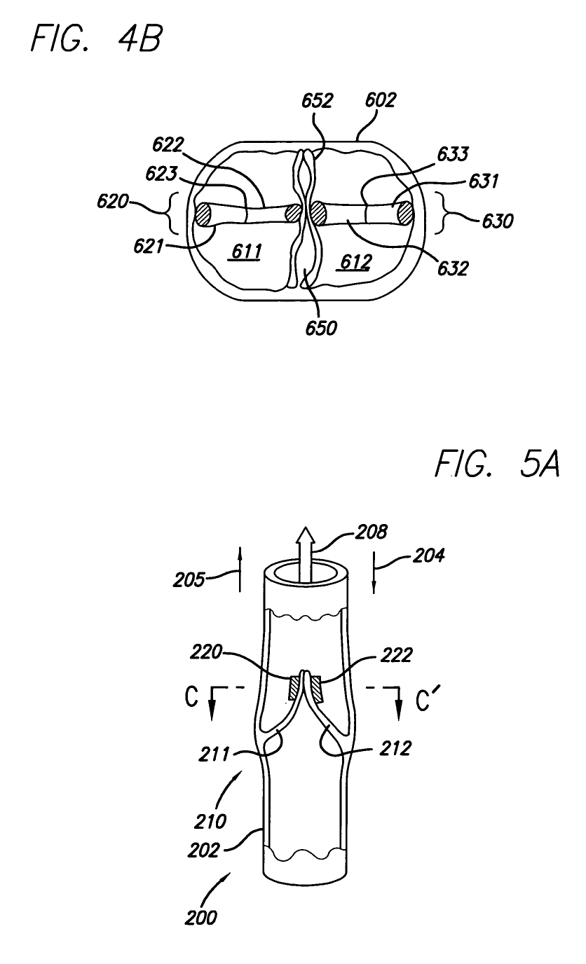

Valves can also be modified by implanting medical devices comprising resilient materials. FIG. 4A and FIG. 4B together show a segment of a vein 600 comprising a venous valve 610. A pair of substantially identical medical devices 620, 630 are deployed within the vein 600. Each medical device 620, 630 is a bent tube of superelastic material that is slightly compressed at a more acute angle within the vein 600 than the configuration each tube would assume without external restraint. The first medical device 620 comprises a first member 621 in contact with a portion of the vein wall 602 and resiliently joined by a first bend 623 to a second member 622 that contacts a portion of a first leaflet 611. Similarly, the second medical device 630 comprises a third member 631 in contact with a portion of the vein wall 602 and resiliently joined by a first bend 633 to a second member 632 that contacts a portion of a second leaflet 612. FIG. 4B shows the D-D′ cross section of FIG. 4A. The pair of medical devices 620, 630 are opposably positioned within the lumen of the vein 600.

Optionally, the ends of each medical device can include a magnet material. For example, the first medical device 620 comprises a distal magnet 625 at one end of the bent tube and a proximal magnet 624 at the proximal end. The distal magnet 625 and the proximal magnet 624 can be oriented to provide a mutually repulsive force to promote the closure of the valve leaflets. Similarly, the second medical device 630 includes a proximal magnet 634 and a distal magnet 635 that are oriented to mutually repel each other. Alternatively, the proximal and distal magnets can be oriented to attract each other, counteracting the expansion of the superelastic material into the vessel wall and the valve leaflet.

The amount of mechanical resilience of the medical devices 620, 630 can be calibrated to the intended function within the vein 600. Preferably, the medical devices 620, 630 maintain sufficient outward pressure against the wall of the vein 602 and at least one leaflet 611, 612 to keep the medical devices 620, 630 from migrating or changing orientation within the vein 600. In one aspect, the medical devices 620, 630 are sufficiently resilient to releasably close the leaflets 611, 612 to permit the valve 610 to open in response to fluid flow in a first direction 605, while promoting closure of the valve 610 when fluid flows in the opposite direction 604. In another aspect, shown in FIG. 4B, the medical devices 620, 630 maintain the central portion of the leaflets 611, 612 together, thereby diverting fluid flow in the first direction 605 through two adjacent valve orifices 650, 652, while preventing fluid flow in the opposite direction 604. The medical devices 620, 630 are preferably formed from a resilient material such as Nitinol (a superelastic NiTi alloy). While this embodiment describes medical devices with a tubular configuration, any suitable shape can be deployed, including disks, cylinders, spheres, laminar sheets, and others. Optionally, portions of the medical devices 620, 630 can be adapted to attach to portions of the body vessel or leaflet, for example by including structural features such as barb structures, or applying adhesives or other sealing means including tissue welding or UV-activated adhesives, or using RF energy emitted from a catheter probe.

Medical devices for releasably closing or connecting portions of valve leaflets may be made of any resilient material known in the art including polymeric materials, metals, ceramics and composites. Where the resilient material is made of metal, the metal may be stainless steel, cobalt-chromium, elgiloy, tantalum or other plastically deformable metals. Other suitable metals include superelastic shape memory metals such as the NiTi alloy NITINOL™.

Incorporation of Bioactive Agents

The implantable medical device can optionally comprise one or more bioactive agents. Medical devices comprising an antithrombogenic bioactive agent are particularly preferred for implantation in areas of the body that contact blood. An antithrombogenic bioactive agent is any therapeutic agent that inhibits or prevents thrombus formation within a body vessel. The medical device can comprise any suitable antithrombogenic bioactive agent. Types of antithrombotic bioactive agents include anticoagulants, antiplatelets, and fibrinolytics. Anticoagulants are bioactive agents which act on any of the factors, cofactors, activated factors, or activated cofactors in the biochemical cascade and inhibit the synthesis of fibrin. Antiplatelet bioactive agents inhibit the adhesion, activation, and aggregation of platelets, which are key components of thrombi and play an important role in thrombosis. Fibrinolytic bioactive agents enhance the fibrinolytic cascade or otherwise aid is dissolution of a thrombus. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin.

Further examples of antithrombotic bioactive agents include anticoagulants such as heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, Cl-1083, JTV-803, razaxaban, BAY 59-7939, and LY-51,7717; antiplatelets such as eftibatide, tirofiban, orbofiban, lotrafiban, abciximab, aspirin, ticlopidine, clopidogrel, cilostazol, dipyradimole, nitric oxide sources such as sodium nitroprussiate, nitroglycerin, S-nitroso and N-nitroso compounds; fibrinolytics such as alfimeprase, alteplase, anistreplase, reteplase, lanoteplase, monteplase, tenecteplase, urokinase, streptokinase, or phospholipid encapsulated microbubbles; and other bioactive agents such as endothelial progenitor cells or endothelial cells.

An antithrombogenic bioactive agent can be incorporated in or applied to portions of the implantable medical device by any suitable method that permits adequate retention of the bioactive agent material and the effectiveness thereof for an intended purpose upon implantation in the body vessel. The configuration of the bioactive agent on or in the medical device will depend in part on the desired rate of elution for the bioactive. Bioactive agents can be coated directly on the medical device surface or can be adhered to a medical device surface by means of a coating. For example, an antithrombotic bioactive agent can be blended with a polymer and spray or dip coated on the device surface. A bioactive agent material can be posited on the surface of the medical device and a porous coating layer can be posited over the bioactive agent material. The bioactive agent material can diffuse through the porous coating layer. Multiple porous coating layers and or pore size can be used to control the rate of diffusion of the bioactive agent material. The coating layer can also be nonporous wherein the rate of diffusion of the bioactive agent material through the coating layer is controlled by the rate of dissolution of the bioactive agent material in the coating layer. The bioactive agent material can also be dispersed throughout the coating layer, by for example, blending the bioactive agent with the polymer solution that forms the coating layer. If the coating layer is biostable, the bioactive agent can diffuse through the coating layer. If the coating layer is biodegradable, the bioactive agent is released upon erosion of the biodegradable coating layer. Bioactive agents may be bonded to the coating layer directly via a covalent bond or via a linker molecule which covalently links the bioactive agent and the coating layer. Alternatively, the bioactive agent may be bound to the coating layer by ionic interactions including cationic polymer coatings with anionic functionality on bioactive agent, or alternatively anionic polymer coatings with cationic functionality on the bioactive agent. Hydrophobic interactions may also be used to bind the bioactive agent to a hydrophobic portion of the coating layer. The bioactive agent may be modified to included a hydrophobic moiety such as a carbon based moiety, silicon-carbon based moiety or other such hydrophobic moiety. Alternatively, the hydrogen bonding interactions may be used to bind the bioactive agent to the coating layer.

Optionally, the bioactive agent can be combined with a bioabsorbable polymer and mixed with, or deposited on, the magnetic remodelable material. Bioabsorbable materials absorb into the body after a period of time. The period of time for the absorption may vary, but is typically sufficient to allow adequate tissue growth at the implant location to provide a desirable modification of a valve. The bioabsorbable material can be polylactic acid, polyglycolic acid, polydioxanone, or a copolymer or mixture thereof. A non-bioabsorbable polymer may also be mixed with, or coated over, the magnetic remodelable material, such as polytetrafluoroethylene (PTFE, including expanded PTFE (ePTFE), polyalkylmethacrylates such as polymethlymethacrylate (PMMA), and parylene C. Phosphatidylcholine, phosphorylcholine and endothelial progenitor cells are other examples of biological molecules that can also be coated on or mixed with the magnetic material.

Methods for Modification of a Body Valve

In a fourth embodiment, methods of modifying a valve in the body are provided. Preferably, a valve in a body vessel is modified by desirably promoting the opening or closing of moveable portions of the valve, such as opposable leaflets of a venous valve or a heart valve. Methods of modifying a valve in a body vessel can comprise the step of implanting a magnetic material within the body vessel. The implanted material can comprise a magnet material in combination with a remodelable material, synthetic polymer material or biomaterial. Any suitable remodelable material, synthetic biocompatible material, biomaterial or combination thereof can be combined with a magnetic material to form an implantable medical device. Preferred remodelable materials, such as SIS, are discussed above. Biocompatible polyurethane polymers, including polyureaurethane polymers, are one category of a preferred synthetic polymer. Collagen is one example of a preferred biomaterial.

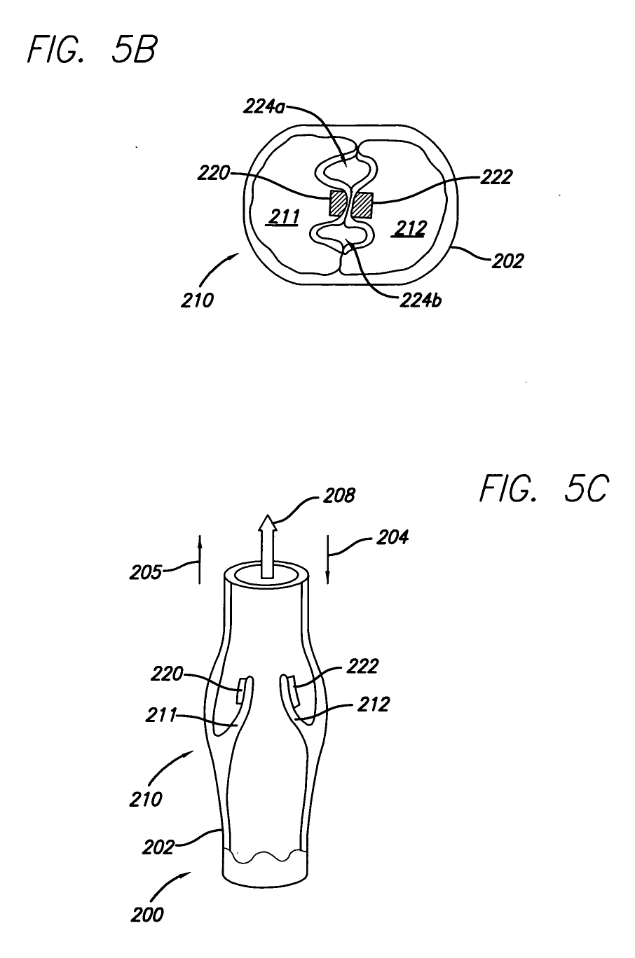

The method of modifying a valve in a body preferably comprises the step of intraluminally implanting a means for modifying a valve in a body vessel. Valves can be modified in any suitable manner. Referring to FIG. 5A, a vein segment 200 comprising an incompetent venous valve 210 contiguously attached to the vein wall 202 is shown. A first medical device 220 is a thin laminar disk comprising magnetic material, similar to the device 100 shown in FIG. 2A and FIG. 2B, is attached to a first valve leaflet 211. The first medical device 220 is opposably positioned with respect to a second medical device 222 that is attached to a second valve leaflet 212. The second medical device 222 is similar to the first medical device 222, except that the first medical device 220 and the second medical device 222 have magnetic portions that are oriented to attract each other across the valve orifice. After implantation as shown, the pair of medical devices 220, 222 permit fluid flow 208 in a first direction 205 while substantially preventing retrograde flow in the opposite direction 204. In a vein 200, the first direction 205 is toward the heart. The laminar disk 220 preferably optionally comprises magnetic microparticles dispersed in a polyureaurethane synthetic polymer.

In one aspect, a means for connecting portions of a valve is implanted. For example, valves can be modified by connecting portions of the valve to prevent movement of portions of the valve relative to each other. In one particular aspect, central portions of opposing venous valve leaflets are strongly magnetically attracted to one another and joined to close a portion of the valve orifice. This permits adjacent opposable leaflet portions to function independently as one way valves. The magnetic attraction can be strong enough to not permit separation of the joined portions of the valve leaflets in response to fluid flow in the body vessel in any direction. Portions of opposable valve leaflets can also be connected to redirect fluid flow around the point of connection. In FIG. 5A, the pair of medical devices 220, 222 join and maintain a portion of the first leaflet 211 in connection with the second leaflet 212. FIG. 5B shows a cross section 210 along the plane C-C′ in FIG. 5A. An attractive magnetic field between the first medical device 220 and the second medical device 222 keeps the pair of medical devices 220, 222 connected, maintaining the central portions of the leaflets 211, 212 together, regardless of the direction of fluid flow. The magnetic attraction between the medical devices 220, 222 is stronger than the force exerted by fluid flow 208 in the first direction 205 to separate the leaflets 211, 212 and open the valve. When fluid flows in the first direction 205 in FIG. 5A, the fluid flow 208 is bifurcated to pass through a first valve orifice 224a and a second valve orifice 224b on either side of the pair of the static magnetic medical devices 220, 222. The first valve orifice 224a and the second valve orifice 224b operate independently to competently regulate fluid flow 208 in a competent, substantially unidirectional manner.

In another aspect, a means for releasably attracting independently moveable opposable portions of a valve is implanted in a body vessel. Magnetic devices can be implanted on opposable portions of two or more valve leaflets to releasably attract the opposable portions of the valve leaflets to promote movement of the portions of the valve with respect to each other and promote closure of the valve orifice. For example, a first magnetic device can be attached to first valve leaflet, and a second magnetic device can be attached to a second valve leaflet to releasably attract the two valve leaflets toward a closed valve position to permit unidirectional fluid flow through the valve by promoting the closure of the leaflets in response to retrograde fluid flow. In another aspect, shown in FIG. 5C, the first medical device 211 and the second medical device 222 releasably bias the first leaflet 211 toward the second leaflet 212. The magnetic fields of the first medical device 220 and the second medical device 222 are oriented to weakly attract each other across the space between the first leaflet 211 and the second leaflet 212, permitting the leaflets 211, 212 to separate in response to fluid flow 208 in the first direction 205, and promoting closure of the leaflets 211, 212 in response to retrograde flow in the opposite direction 204.

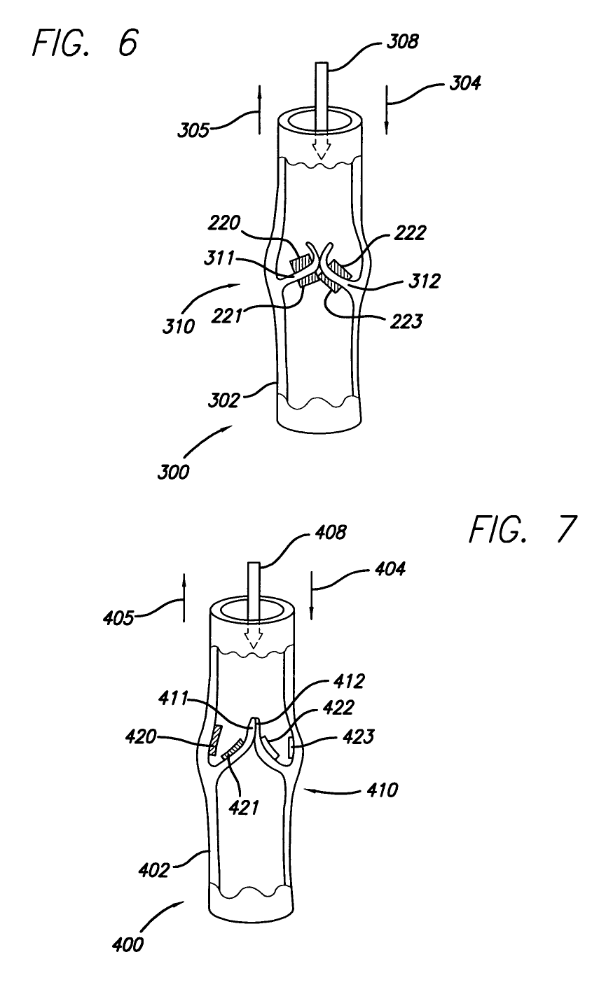

One or more medical devices can also be implanted to modify fluid flow within a branched body vessel network by occluding fluid flow in one segment of the body vessel network. Implantation of medical devices to occlude a vein segment may be desirable, for example, to redirect fluid flow away from a vessel containing incompetent valves and toward other body vessels with more competent valves. In FIG. 6, a valve segment comprises an incompetent valve 310 having a first leaflet 311 and a second leaflet 312. A first pair of medical devices 220, 221 comprising magnetic material are joined together by strong magnetic attraction across the first leaflet 311. Similarly, a second pair of medical devices 222, 223 also comprising magnetic material are joined together by strong magnetic attraction across the second leaflet 312. The first pair of medical devices 221, 222 and the second pair of medical devices 222, 223 are configured and oriented to maintain the valve 310 completely closed and prevent fluid flow 308 in either the first direction 305 or the second direction 304, thereby occluding the vein segment 300.

One or more medical devices can be implanted in contact with the wall of a body vessel. In FIG. 7, competent valve function is restored to an incompetent bileaflet valve 410 within a vein segment 400 by implanting two pairs of magnetic laminar medical devices comprising magnetic material, similar to the medical device 100 in FIG. 2A and FIG. 2B. A first pair of magnetic laminar devices 420, 421 are oriented to repel each other and thereby move a first leaflet 411 toward the closed position. A first medical device 420 is attached to the vein wall 402 and is oriented to magnetically repel a second medical device 421 that is attached to the first leaflet 411. Similarly, a second pair of magnetic laminar devices 422, 423 are oriented to repel each other to move a second leaflet 412 toward the closed position. A third medical device 423 is attached to the vein wall 402 and is oriented to magnetically repel the fourth medical device 422 that is attached to the second leaflet 412. As a result, fluid flow 408 is permitted in a first direction 405 but substantially prevented in the opposite direction 404.

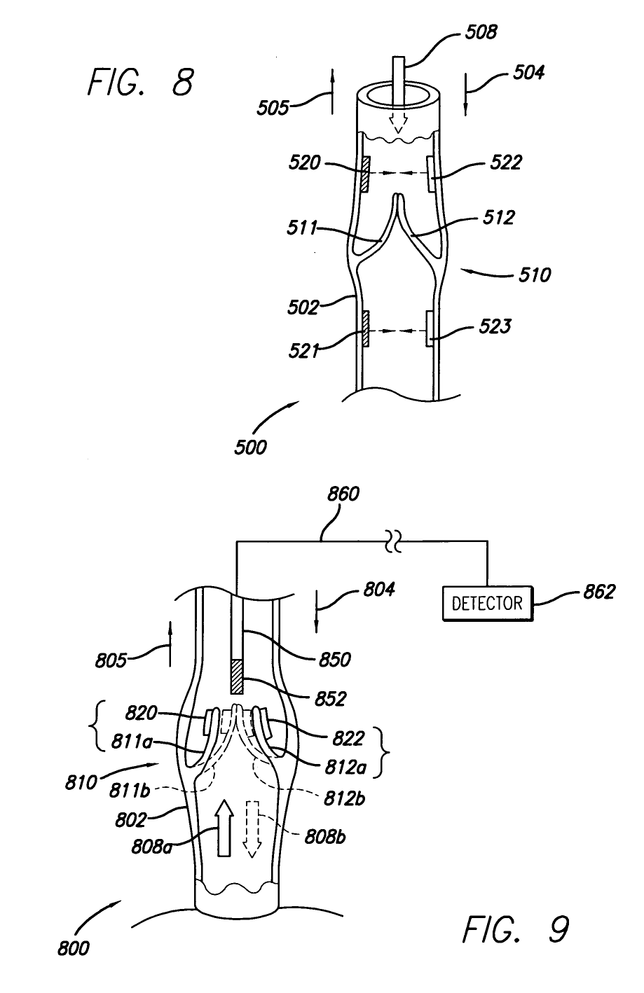

FIG. 8 shows restoration of valve function is restored of a bileaflet valve 510 within a body vessel 500 after implanting two pairs of strongly attracted magnetic medical devices to the interior wall 502 of the body vessel 500. A first pair of magnets 520, 522 are anchored to opposite portions of the interior wall 502 of the body vessel 500 in a first direction 505 from the bileaflet valve 510. The first magnet 520 is strongly attracted to the second magnet 522 across the lumen of the body vessel 500, thereby narrowing the caliber of the body vessel lumen. Similarly, a second pair of magnets 521, 523 are positioned at a position in a second direction 504 along the body vessel 500 from the valve 510. The third magnet 521 is strongly attracted to the fourth magnet 523, while both magnets 521, 523 are strongly attracted to each other so as to narrow the caliber of the lumen of the body vessel 500 therebetween. The narrowing of the body vessel by the first pair of magnets 520, 522 and the second pair of magnets 521, 523 draws a first leaflet 511 closer to a second leaflet 512 so as to restore or improve the function of the valve 510.

Methods of Valve Monitoring

In a fifth embodiment, methods of monitoring the movement of a valve within a body vessel are provided. In one aspect, a method of monitoring the movement of a valve in a body vessel comprises the steps of: implanting a detectable material in moveable contact with a leaflet of the valve, and detecting the movement of the detectable material. A preferred method of monitoring the movement of a valve in a body vessel preferably comprises the steps of: implanting a detectable magnetic material in moveable contact with a venous valve leaflet, and detecting the movement of the magnetic material. Preferably, the implantable detectable material comprises a magnetic material that is in operative communication with a moveable portion of a valve, such as a venous valve leaflet, so that movement of the leaflet generates a detectable signal. For example, the detectable material can be a thin magnetic laminar material that is joined to and moveable with a venous valve leaflet in response to blood flow through a vein. Movement of the venous valve leaflet is preferably monitored by detecting movement of the magnetic material attached to the venous valve leaflet using a detection means. The detection means can be one or more detection structures capable, individually or in combination, of detecting movement of a signaling device. Preferably, the detection structure is a detecting structure placed outside the body and adapted for detection of the movement of the signaling device. Alternatively, a catheter probe placed within a body vessel as a detecting structure.

In FIG. 9, a first remodelable magnetic material 820 is attached to a first leaflet 811, and a second remodelable magnetic material 822 is attached to a second leaflet 812 of a venous valve 810 within a vein 800 segment. The venous valve 810 functions competently and opens and closes in response to the direction of fluid flow. When fluid flows in a first direction 808a, the first leaflet 811 and the second leaflet 812 are in an open position, 811a, 812a to allow fluid flow. When fluid flows in a second opposite direction 808b, the first leaflet 811 and the second leaflet 812 are in a closed position, 811b, 812b to block fluid flow. Motion of the remodelable magnetic material 820, 822 on the leaflets 811, 812 creates a signal that can be detected and correlated to valve function. A catheter probe 850 inserted in the vein 800 comprises a detection portion 852 adapted to detect movement of the remodelable magnetic material 820, 822 on the leaflets 811, 812. The catheter probe 850 communicates 860 with a detection means 862 by any suitable means, such as an electronic signal detection process.

The detectable material can be any suitable material, including without limitation: a remodelable magnetic material, magnetic particles of any suitable size, or fluoroscopic compounds. For example, Brandl et al., “Detection of Fluorescently Labeled Microparticles in Blood,” Blood Purif., February 10;23(3):181-189 (2005), incorporated herein by reference, describes cellulose microsphere particles having diameters of 1-20 μm detected with an optical detection system, both ferromagnetic and fluorescence labeled. By illuminating a small volume of blood with a catheter probe, using an excitation wavelength (590 nm) of the fluorescence marker, the particles can be detected by their emission light at 620 nm. Another relevant technology is disclosed by Mirkin et al., “Nanoparticle based bio bar codes for the ultrasensitive detection of proteins,” Science, 2003 Sep. 26; 301(5641):1884-6, incorporated herein by reference.

Various means for monitoring the movement of valves within a body vessel, and methods of using the same, are also provided. The means for detecting the movement of the detectable material can be a catheter probe adapted to detect movement of magnetic material, or a device for optical detection of fluoroscopic compounds. Preferably, the means for monitoring the movement of valves comprises a magnetic material. The implantable monitoring device can be implanted in operative communication with a valve in a body vessel so that movement of the implantable monitoring device can be correlated with movement of the valve. For example, the implantable monitoring device can be a thin flexible laminar sheet that is attached to and moves with one surface of a venous valve after implantation.

The detectable material can be one or more signaling devices of any size and shape that are configured for implantation within a body vessel and are individually or together capable of providing a detectable signal in response to movement of a valve within the body vessel. The movement of a valve in a body vessel can be monitored by detecting the movement of one or more signaling devices implanted within a body vessel.

In one embodiment, a signaling structure comprises a magnetic material that can be implanted with at least one surface of the signaling structure moveable in response to the movement of a venous valve leaflet in situ. Alternatively, the signaling structure can be implanted with a first surface in contact with a venous valve leaflet and a second surface in contact with a portion of the interior vein wall. Still other embodiments provide signaling structures implanted with a surface contacting the interior of a body vessel, without contacting a valve leaflet within the body vessel.

An implantable medical device comprises a bioabsorbable material, allowing for a temporary monitoring of valve leaflets, or closure of two or more valve leaflets against each other, within a body vessel. Optionally, a magnet material can be coated on or impregnated in at least a portion of the bioabsorbable material. Any suitable bioabsorbable material that is gradually absorbed by the body can be used. A bioabsorbable medical device can provide an initial level of closure of a valve leaflet upon implantation and then provide a diminished or negligible valve closure function to the valve upon absorption of the bioabsorbable material.

Implantable devices can also optionally comprise radiopaque markers or a radiopaque coating or impregnation, for example to assist in visualization of the material during a non invasive procedure. Any suitable radiopaque substance, or combination of radiopaque substances, can be used. For example, radiopaque substances containing tantalum, barium, iodine or bismuth in powder form can be coated upon or incorporated within a material used in an implantable medical device.

Magnetic Particles

In a sixth embodiment, a method for modifying or monitoring the movement of a body valve comprises attaching magnetic particles to portions of a body vessel or a valve within the body to modify a valve within the body. For example, magnetic particles adapted to attach to portions of the surface of the body vessel or valve can be implanted using a catheter. Any suitable form of magnetic particle can be implanted. The size of the magnetic particles is selected to provide a desired amount of magnetic attraction or repulsion within a body vessel, or to promote remodeling of remodelable material within the body. Magnetic particles can further comprise binding sites to promote attachment to portions of a valve within a body vessel. Magnetic beads such as Dynabeads™, with optionally modified surface properties, can be used as magnetic particles, for instance as described by Tiwari et al., “Magnetic beads (Dynabead) toxicity to endothelial cells at high bead concentration: implication for tissue engineering vascular prosthesis,” Cell. Biol. Toxicol., 19(5), 265-272 (October 2003), which is incorporated by reference. Another suitable magnetic particle can be a 1-2 μm polystyrene particle coated with a mixture of magnetic iron oxide (magnetite) and polystyrene, such as the paramagnetic particles sold under the tradename SPHERO™ Magnetic Particles (Spherotech, Inc., Libertyville, Ill.); the polystyrene polymer combined with the magnetite can optionally be cross linked to increase the surface area and magnetite content. Another suitable source of a magnetic material are magnetic ferrofluids comprising nanoparticles (ca. 1-100 nm) of iron oxides in a stable colloidal suspension in water at about 1.7-5.0 v %, such as the ferrofluid sold under the tradename Pure Precision™ available from FerroTec containing a mixture of 10 nm particles of Fe₃O₄ and γ-Fe₂O₃ iron oxides.

Magnetic particles can optionally further comprise biomolecules such as glucoamylase immobilized on the particle surface. One such microparticle is the magnetic microparticle of polyethyleneimine coated magnetite optionally crosslinked with glutaraledhyde and optionally derivatized with adipic dihydrazide, as described by B R Pieters et al., “Glucoamylase immobilization on a magnetic microparticle for the continuous hydrolysis of maltodextrin in fluidized bed reactor,” Appl. Biochem. Biotechnol., 23, 37-53 (January-March 1992).

Another suitable magnetic particle source includes the amine-terminated magnetic beads sold under the trade name BcMag (BioClone Inc., San Diego, Calif.) are supplied as an aqueous suspension of magnetic iron oxide particles having primary amino groups on the surface (1-5 μm beads, 50 mg/ml in 1 mM EDTA, pH 7.0). The BcMag bead can be attached to a protein in an injectable fluidized particulate ECM by preparing a solution comprising 30-100 mg of the fluidized ECM and 10 mL of a suitable coupling buffer and mixed, per manufacturer's directions. The injectable particulate construct of the ECM and magnetic particle can be injected locally within a body vessel to adhere the magnetic material within a body vessel. Any suitable magnetic material can be combined with a suitable molecular moiety, such as primary amino groups, to bind to protein in a fluidized ECM material.

Superparamagnetic iron oxide nanoparticles, optionally coated with a bioactive, are another example of a suitable magnetic particle, for example as described by Gupta et al., “Receptor mediated targeting of magnetic nanoparticles using insulin as a surface ligand to prevent endocytosis,” IEEE Trans Nanobioscience, 2(4), 255-261 (December 2003) and Gupta et al., “Surface modified superparamagnetic nanoparticles for drug delivery: interaction studies with human fibroblasts in culture,” J. Mater. Sci. Mater. Med., 15(4), 493-496 (April 2004), both of which are incorporated by reference herein. Magnetic particles optionally embedded in a microgel or hydrogel can also be used, for example as described by Pich et al., “Temperature sensitive hybrid microgels with magnetic properties,” Langmuir, 20(24), 10706-10711 (November 2004), incorporated by reference herein.

Magnetite nanoparticles and human aortic endothelial cells (HAECs) impregnated with magnetite nanoparticles at a concentration of about 30-50 pg per cell are two more examples of suitable magnetic particles, both of which are described by A. Ito et al., “Tissue engineering using magnetite nanoparticles and magnetic force: heterotypic layers of cocultured heaptocytes and endothelial cells,” Tissue Eng., 10 (5-6), 833-840 (May June 2004), incorporated herein by reference.

Magnetic particles can also be spherical polyacrylamide/magnetite composite beads such as those described by Cocker et al., “Preparation of magnetically susceptible polyacrylamide/magnetite beads for use in magnetically stabilized fluidized bed chromatography,” Wiley Interscience Journal, 1996, incorporated herein by reference.

Another example of a suitable particle structure is a hydrophobic magnetic Ni-polytetrafluoroethylene (Ni-PTFE) microparticle disclosed in Zhi Z L, et al., “Multianalyte immunoassay with self assembled addressable microparticle array on a chip,” Anal. Biochem., 2003 Jul. 15;318(2):236-43, which is incorporated herein by reference. Preferably, the microparticle structure further comprises surface binding chemical groups to promote attachment to the inner wall of a body vessel.

Delivery Systems

Medical devices can be implanted within a body vessel using transcatheter implantation techniques known in the art. In some embodiments, one or more valve monitoring means, one or more valve closure means, or one or more valve connecting means, or both, can be implanted within a body vessel using a catheter. Examples of valves that can be monitored, releasably closed or permanently connected after implantation of one or more medical devices (e.g., by magnetic attraction or repulsion, or by exerting resilient force against a valve surface) include a natural valve in the body vessel, such as a native venous valve, or a previously implanted prosthetic valve. In some embodiments, medical devices comprising a magnetic material can be implanted inside a vein such that at least one surface of the medical device contacts a venous valve leaflet, or at least one medical device surface contacts the interior wall of the vein. In some embodiments, medical devices comprising a resilient material is implanted in contact with a venous valve leaflet and an adjacent region of the interior wall of the vein, for example bridging a leaflet sinus region.

In one aspect, a remodelable material, a synthetic polymer material or a biomaterial comprising a magnetic material can be delivered within a body vessel. The remodelable material, synthetic polymer material or biomaterial magnetic material can be fashioned into any suitable shape, size or construction for placement in any suitable location along a valve leaflet or body vessel wall. For example, a remodelable material, synthetic polymer material or biomaterial may be combined with a magnetic material and attached to a portion of a valve leaflet using a suitable glue or bonding agent. Alternatively, the remodelable material, synthetic polymer material or biomaterial may be secured to portions of two or more valve leaflets. In still other embodiments, a remodelable material, synthetic polymer material or biomaterial combined with a magnetic material may be secured to a valve leaflet using electrodes equipped with an energy source, such as radiofrequency (RF) energy, emitted from a portion of a catheter within the body vessel.

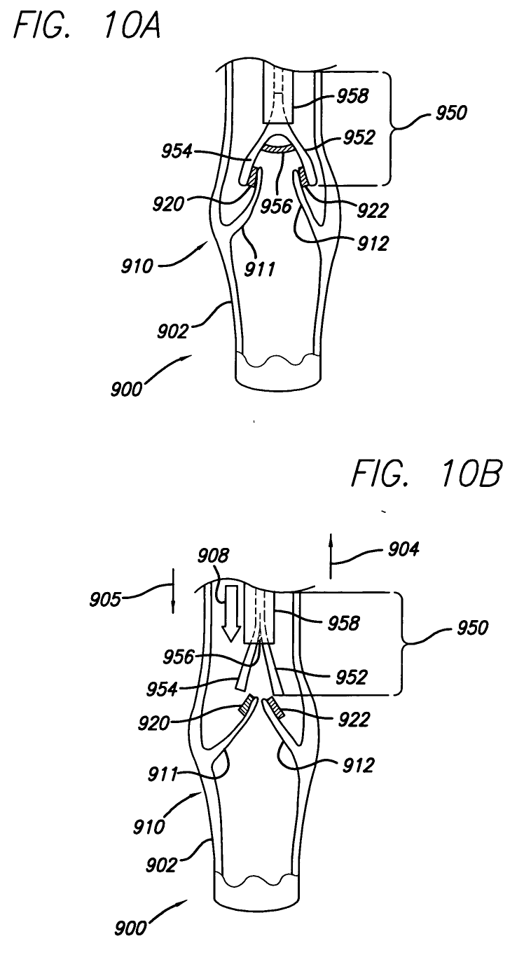

FIG. 10A and FIG. 10B show a portion of a catheter delivery system for a pair of devices comprising remodelable magnetic material. The catheter delivery system 950 is positioned at a site of treatment within the lumen of a vein 900. The distal end of the catheter delivery system 950 comprises a first arm 952 exerts outwardly force away from a second arm 954. When unrestrained, the first arm 952 and the second arm 954 form a “Y” shape with the more proximal portion of the catheter delivery system 950. A spring means 956, such as a spring or repulsively oriented magnets, is optionally included in the catheter delivery system 950 to outwardly push the first arm 952 and the second arm 954 away from each other.

The catheter delivery system 950 further comprises a flexible outer sheath 958 that compresses the first arm 952 and the second arm 954 toward each other to retract the delivery system. In one aspect, the first arm 952 and the second arm 954 comprise a self expanding material. Prior to implantation, a pair of substantially identical remodelable magnetic implantable laminar devices 920, 922 are reversibly joined to arms 952, 954 of the catheter delivery system. In operation, the catheter delivery system 950 is inserted into a body vessel with the outer sheath 958 covering the arms 952, 954 in a compressed state. At the point of treatment, the outer sheath 958 is moved away from the distal end of the arms 952, 954, thereby uncovering the arms 952, 954 and allowing for expansion of the arms 952, 954. The remodelable magnetic implantable laminar patches 920, 922 are positioned over a first valve leaflet 911 and a second valve leaflet 912 as shown in FIG. 10A. The remodelable magnetic implantable laminar patches 920, 922 are then attached to the first valve leaflet 911 and the second valve leaflet 912 in any suitable fashion. For example, by pressing barbs or adhesive in the remodelable magnetic implantable laminar patches 920, 922 into the leaflets. In another aspect, the arms 952, 954 are heated or provide RF energy to join the tissue of the remodelable magnetic implantable laminar patches 920, 922 to the valve leaflets 911, 912. After attachment of the remodelable magnetic implantable laminar patches 920, 922 to the leaflets 911, 912, in FIG. 10B, the outer sheath 958 is translated toward the distal end of the arms 952, 954 to compress and cover the arms 952, 954 and the catheter delivery system 950 is then removed from the body vessel. Preferably, the implanted remodelable magnetic implantable laminar patches 920, 922 restore or improve the function of a valve 910, so that fluid flow 908 is permitted in a first direction 904 but substantially prevented in an opposite direction 905.

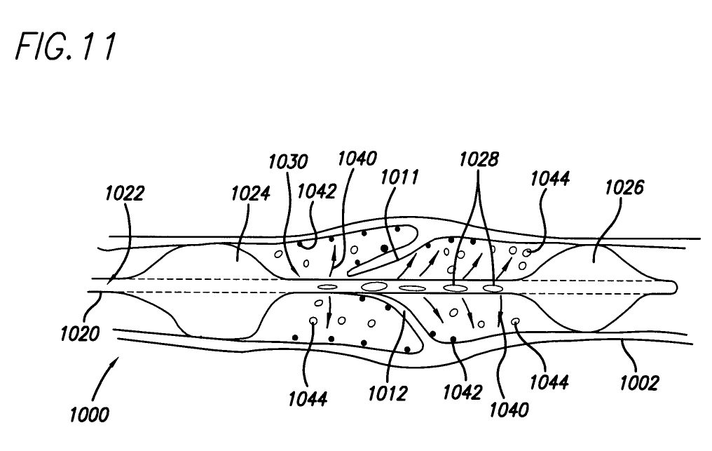

Delivery systems for implanting detectable material within a body vessel that can be monitored within the body vessel are also provided. FIG. 11 shows a first delivery system for delivering a particulate detectable material within a portion of a body vessel. A catheter delivery system 1020 comprises a proximal balloon 1024 a distal balloon 1026 and a plurality of delivery ports 1028 positioned therebetween. The catheter delivery system 1020 is inserted within a body vessel 1002 to a point of treatment, where the proximal balloon 1024 and the distal balloon 1026 are inflated to create a contained lumen region 1030. The contained lumen region 1030 comprises a first leaflet 1011 and a second leaflet 1012 of a body valve to be monitored. A detectable material 1044, such as magnetic microparticles, is then ejected through the plurality of delivery ports 1028 and contained within the contained lumen region 1030. The detectable material 1044 comprises surface binding moieties that adhere to the wall of the body vessel 1002, and a percentage of the detectable material 1044 binds to the interior surface of the body vessel 1002 and the valve therein as bound detectable material 1042. Residual detectable material 1044 can be drawn out of the contained lumen region 1030 through the delivery ports 1028, leaving the bound detectable material 1042 within the body vessel 1002. The balloons 1024, 1026 can then be deflated and the catheter delivery system 1020 removed from the body vessel 1002.

Materials and devices can be implanted within a body vessel at any suitable orientation or position. Preferably, a device can be implanted in contact with a portion of a valve within the body. In some aspects, an implantable device comprising a magnetic material and a remodelable material, synthetic polymer material or biomaterial is transluminally implanted within a body vessel. In one aspect, a magnetic remodelable material, synthetic polymer material or biomaterial is preferably delivered using a catheter-based delivery system within a body vessel. In another aspect, an implant is positioned in contact with a portion of a valve in a body vessel, such as a valve leaflet of a venous valve or heart valve. In another aspect, two or more devices comprising magnetic material can be implanted, simultaneously or sequentially, in contact with two or more portions of a valve or body vessel that are moveable with respect to one another. For example, a first magnetic remodelable material, synthetic polymer material or biomaterial can be implanted in contact with a first valve leaflet; a second magnetic remodelable material, synthetic polymer material or biomaterial can preferably be implanted in contact with a second valve leaflet that is opposable to the first leaflet, or in contact with a portion of the wall of the body vessel. In other aspects, a device comprising a resilient material such as a superelastic NiTi alloy is implanted in contact with a moveable portion of a valve. In one aspect, a device comprising a resilient alloy is implanted with a first surface contacting a valve leaflet joined to a second surface contacting a portion of the vessel wall. Preferably, the first surface of the device hingeably moves relative to the second surface.

Methods of treating a patient for conditions such as venous valve insufficiency are also provided. In one aspect, a method of treatment comprises the step of determining a location for implanting a medical device or determining an inner diameter of a body vessel at a point of treatment. This step can be accomplished by any appropriate vessel sizing technique known in the art. A second step in a method of treatment preferably comprises selecting a suitable medical device for implantation. A suitable medical device can be chosen based on factors such as the ability of the body vessel to alter its shape to accommodate the implanted medical device. Preferably, the medical device with a dimension appropriate for the treatment based upon the inner diameter of the body vessel at the point of treatment. A third preferred step in a method of treatment is implanting the medical device at a point of treatment in a body vessel, for example using a catheter delivery system.

One preferred method is a venous catheterization to repair an incompetent venous valve by implanting one or more medical devices to improve venous valve function. One or more medical devices are delivered to a point of treatment near the incompetent venous valve from the distal portion of a catheter delivery device. Preferably, access to the vein can be established at any suitable location on the subject's body, such as the neck, ankle or knee. Alternatively, access can be established surgically, for example by performing a cutdown to a suitable location. After access is achieved to the vein, a medical device can be delivered by translating the distal tip of the catheter to the point of treatment.

While certain embodiments disclosed herein relate to the modification or modification of venous valve function within a body vessel, the invention is not limited to venous valve modification or monitoring. Non-limiting examples of suitable valves include any valves with leaflets, such as bicuspid calf valves and tricuspid valves such as heart valves. Embodiments are also provided that relate to monitoring, or modifying the function of previously implanted prosthetic valves in any body vessel.

The embodiments described herein can be equally applied to other locations and lumens in the body, such as, for example, coronary, vascular, nonvascular and peripheral vessels, ducts, and the like, including but not limited to cardiac valves, venous valves, valves in the esophagus and at the stomach, valves in the ureter and/or the vesica, valves in the biliary passages, valves in the lymphatic system and valves in the intestines.

What is claimed is:

1. A composition comprising a magnetic material attached to a remodelable material.

2. The composition of claim 1, where the remodelable material is an extracellular matrix material.

3. The composition of claim 1, where the remodelable material is small intestine submucosa.

4. The composition of claim 1, where the magnetic material comprises a material selected from the group consisting of: iron oxide, magnetite, samarium cobalt, magnetic ferrite, ferrite, strontium ferrous oxide, NdFeB, SmCo, aluminum, nickel, cobalt, copper, iron and titanium or combinations thereof.

5. The composition of claim 1, further comprising a particle comprising the magnetic material, wherein the particle has a mean size of between about 10 nm and 100 μm.

6. The composition of claim 5, where the particle is coated with the magnetic material and a material selected from the group consisting of: poly(ethylene glycol), polystyrene, methylmethacrylate, polyacrylamide, and polytetrafluoroethylene.

7. The composition of claim 1, further comprising a polyurethane.

8. The composition of claim 7, wherein the polyurethane is cross linked to the remodelable material.

9. The composition of claim 1, wherein the magnetic material has a maximum magnetic field of between about 400 and 2,000 gauss.

10. The composition of claim 1, further comprising a diluent, and where the composition is configured as a suspension of particles attached to the remodelable material and the magnetic material.

11. The composition of claim 1, where the remodelable material is configured as a sheet of extracellular matrix material having the magnetic material attached thereto.

12. A flexible laminar patch comprising: a film of extracellular matrix material having a thickness that is less than any planar dimension of the film, and a magnetic material attached to the extracellular matrix material.

13. The flexible laminar patch of claim 12, wherein the film has a thickness of between about 0.0001 inch and about 0.0050-inch.

14. The flexible laminar patch of claim 12, wherein the film comprises small intestine submucosa.

15. The flexible laminar patch of claim 12, wherein the film comprises a first layer and a second layer, the first layer comprising an extracellular matrix material and the second layer comprising a sheet of the magnetic material.

16. The flexible laminar patch of claim 12, wherein the film comprises a single layer having a mixture of the magnetic material with small intestine submucosa.

17. The flexible laminar patch of claim 12, wherein the magnetic material comprises a material selected from the group consisting of: iron oxide, magnetite, samarium cobalt, magnetic ferrite, ferrite, strontium ferrous oxide, NdFeB, SmCo, aluminum, nickel, cobalt, copper, iron and titanium or combinations thereof.

18. The flexible laminar patch of claim 12, wherein the film further comprises a plurality of particles attached to the magnetic material, wherein the particles have a mean size of between about 10 nm and 100 μm.

19. The flexible laminar patch of claim 18, wherein the film further comprises a polyurethane in contact with the remodelable material.

20. A flexible laminar patch comprising: a film of extracellular matrix material comprising one or more layers and having a thickness of between about 0.0001 inch and about 0.0050-inch, and a magnetic material attached to the extracellular matrix material, the magnetic material comprising a material selected from the group consisting of: iron oxide, magnetite, samarium cobalt, magnetic ferrite, ferrite, strontium ferrous oxide, NdFeB, SmCo, aluminum, nickel, cobalt, copper, iron and titanium or combinations thereof.
